(19) United States
(12) Patent Application Publication
(10) Pub. No.: US 2005/0207995 A1
(43) Pub. Date: Sep. 22, 2005

(54) Methods and compositions for promoting oral health, and polypeptides useful for same

(75) Inventors: Richard L. Gregory, Carmel, IN (US); Diana M. Catt, Mooresville, IN (US)

Correspondence Address: Woodard, Emhardt, Moriarty, McNett & Henry LLP;Bank One Center/Tower, Suite 3700, 111 Monument Circle, Indianapolis, IN, 46204-5137, US

(73) Assignee: Advanced Research and Technology Institute, Inc.

(21) Appl. No.: 10/828,837

(22) Filed: Apr. 21, 2004

Related U.S. Application Data
(63) Continuation-in-part of application No. 10/009,004, now abandoned.
(60) Provisional application No. 60/132,312, filed on May 3, 1999.

(51) Int. Cl. A61K 7/28

(52) U.S. Cl. 424/50

(57) ABSTRACT

Described are methods and compositions for promoting oral health in a host that involve the administration of a 65 kDa protein from S. mutans or a fragment or functional analog thereof, to the oral cavity of the host. The 65 kDa protein exhibits murein hydrolase enzyme activity and in inventive embodiments the enzyme or active fragments can be used to lyse bacteria commonly present in dental plaque or other surfaces of the oral cavity. Methods for controlling dental caries include administering non-immunogenic polypeptides from bacterial adhesions to competitively block bacterial attachment without implicating the immune system of the host. Novel isolated DNA encoding the 65 kDa protein or a fragment thereof, as well as recombinant forms of the protein or fragment, and methods for their production, are also described.

### REFERENCE TO RELATED APPLICATIONS

This application is a continuation-in-part of U.S. patent application Ser. No. 10/009,004 filed Nov. 5, 2001, which is a national stage of International Application Serial No. PCT/US00/11992 filed May 3, 2000 and published in English, which claims the benefit of U.S. application Ser. No. 60/132,312 filed May 3, 1999, each of which is hereby incorporated herein by reference in its entirety.

### BACKGROUND OF THE INVENTION

The present invention resides generally in the field of oral health and compositions and methods that are useful therein. In particular aspects, the present invention relates to a Streptococcus mutans protein that has been shown to have murein hydrolase activity effective to lyse a variety of bacteria that occur in dental plaque, and its use in promoting oral health.

As further background, dental plaque is a heterogeneous mixture of bacterial aggregations embedded within a sticky matrix. Typically, the bacterial composition of plaque ranges from 50 to 70 percent. The matrix is derived from materials such as dead cells, salivary glycoproteins and serum proteins that are laid on a polysaccharide backbone. The bacteria synthesize the polysaccharides for the plaque backbone as a step in their own colonization process. In addition to viable bacteria and the matrix, plaque also usually contains food debris, small numbers of epithelial cells, white blood cells and various other components which are derived from the host and the host's activities.

The formation and development or proliferation of plaque occurs in two stages. The first step may require a base layer of salivary glycoproteins on the tooth's surface as well as on the soft tissue in the oral cavity. This base organic layer, derived from saliva, is adsorbed onto the surface and forms an acquired pellicle. This insoluble acquired pellicle serves as the foundation for supragingival plaque. The second step is the bacterial colonization by “pioneering” bacteria of the acquired pellicle. Once the bacteria have attached to the surface of a structure, they aggregate, develop colonies and plaque begins to form.

There are a variety of different bacterial species that may occur in dental plaques. This variation in the types of bacteria is influenced by diet, salivary components and bacterial interactions, to name a few. The location of the plaque in the oral cavity, the time of the day, age of the patient and the status of the general oral hygiene of the patient all contribute to the implications and consequences of dental plaque and periodontal disease. Consequently, it is not surprising that plaque is a heterogeneous collection of bacterial communities attached to the tooth providing a vast array of biochemical and physiological consequences.

Two major pathological conditions that are correlated with the presence of dental plaque are periodontal disease and dental caries. Periodontal disease in particular results from dental plaque that accumulates at the gingival margin. Generally, two broad classes of periodontal disease have been defined—gingivitis and periodontitis.

Gingivitis is an inflammation of the marginal gingival tissue due to the accumulation of dental plaque. For the most part, gingivitis is characterized by redness, swelling and bleeding of the gingival tissue. The extent and severity of these characteristics indicate the degree to which the disease has progressed. Periodontitis is characterized not only by the inflammation of the marginal gingivae, but also by loss of the attachment of the periodontal ligament, loss of alveolar bone and loss of the epithelial attachment due to apical migration. The pathological consequences of these physiological losses is the formation of a periodontal pocket, which can become infected, and thus be the source of bacterial infiltration into the host. The progression of established gingivitis to an advanced lesion can lay the foundation for periodontitis.

As noted above, there is a cause-and-effect relationship between the presence and quantity of plaque, containing a wide variety of colonized bacterial strains, and periodontal disease and caries. Therefore, by removing or inhibiting the formation of plaque, the extent and severity of periodontal disease and caries can be controlled.

In addition to the more common chemical agents such as chlorhexidine, certain enzymes have been suggested as therapeutic agents for promoting oral health, including for controlling dental plaque. Illustratively, enzymes that decompose glucan, a major component of dental plaque, have been proposed for this purpose. Dextranase (alpha-1,6 glucan hydrolase) is one such enzyme, as disclosed for example in U.S. Pat. No. 3,686,393 to Woodruff et al. The enzyme mutanase (a class of alpha 1,3 glucan 3-glucanohydrolase) has also been proposed, and has been shown to have an inhibitory effect on the formation of dental plaque. See, e.g. U.S. Pat. No. 4,353,891 issued to Guggenheim et al. Combinations of dextranase and mutanase enzymes have also been proposed, potentially combined with other enzymes such as proteases, lipases, and amylases. See, e.g., U.S. Pat. No. 6,254,856 issued to Tsuchiya. As well, U.S. Pat. No. 6,355,228 issued to Fuglsang suggests the use of hybrid polypeptides containing a mutan-binding polypeptide bound to one or more enzymes such as oxidases, peroxidases, proteases, lipases, glucosidases, lipases, esterases, deaminases, ureases and polysaccharide hydrolases.

As noted above, the presence of dental plaque is also correlated to the occurrence of dental caries. A large body of evidence supports that the initiation of caries requires a relatively high proportion of Streptococcus mutans bacteria within dental plaque. These bacteria adhere well to the tooth surface, produce higher amounts of acid from sugars than other bacterial types, can survive better than other bacteria in an acid environment, and produce extracellular polysaccharides from sucrose. When the proportion of S. mutans in plaque is high (for example in the range 2-10%) a patient is at high risk for caries. When the proportion is low (less than 0.1%) the patient is at low risk.

In light of this background there remain needs for methods, substances, and compositions for promoting oral health, including for example for controlling dental plaque and/or inhibiting the development of dental caries. The present invention addresses these needs.

### SUMMARY OF THE INVENTION

In one aspect, the present invention provides a method for controlling dental plaque in the oral cavity of a host. The method involves administering to the oral cavity of the host an effective amount of a polypeptide having murein hydrolase activity against one or more bacterial species occurring in dental plaque of the host. In certain embodiments of the invention, the polypeptide is derived from a Streptococcus mutans bacterium, and can have an amino acid sequence corresponding to the amino acid sequence of SEQ. ID NO. 6 or 8, or an amino acid sequence sufficiently duplicative of an amino acid sequence of SEQ. I.D. NO. 6 or 8, to exhibit murein hydrolase activity.

Still another aspect of the present invention relates to an orally-administerable composition for controlling dental plaque comprising a polypeptide having murein hydrolase activity, and an orally acceptable carrier. The composition can be packaged and labeled for used to control dental plaque, and, in certain embodiments, the polypeptide incorporated in the composition is derived from Streptococcus mutans. The polypeptide can include the amino acid sequence of SEQ. I.D. NO. 6 or 8, or an amino acid sequence sufficiently duplicative of the amino acid sequence of SEQ. I.D. NO. 6 or 8, to exhibit murein hydrolase activity.

In another aspect, the present invention provides methods for controlling dental caries in the oral cavity of a host. The methods comprise administering to the oral cavity of the host an effective caries inhibiting amount of a purified protein having an amino acid sequence corresponding to that shown in SEQ. ID NO. 6 or 8, or an effective polypeptide fragment or functional analog thereof. In such methods, the protein or fragment can be purified from natural sources or recombinantly-produced. Oral administration is desirably accomplished using an oral composition including the protein or fragment and an orally acceptable excipient. The oral composition may be, for example, an aqueous mouthwash or rinse composition, a dentifrice composition, or a chewing gum. Periodic application of the protein or fragment, for example at least once daily, will be preferred.

Another aspect of the invention concerns methods for controlling dental caries in a host which comprises periodically administering to the oral cavity of the host a substance that interferes with the accumulation, mobility, persistence or other activity of S. mutans within dental plaque. In certain forms of the invention, the substance may be or may include an antibody to a murein hydrolase enzyme of S. mutans. In other forms of the invention, the substance may be or may include a substrate for a murein hydrolase enzyme of S. mutans, for instance including a peptidoglycan or peptidoglycan fragment, that permanently or competitively interferes with the murein hydrolase enzyme. In these and other related embodiments, the enzyme may have an amino acid sequence corresponding to that in SEQ. ID NO. 6 or 8, or sufficiently duplicative thereof to exhibit murein hydrolase activity. In still other aspects of the invention, such methods for controlling dental caries may be achieved by the periodic administration of a non-immunogenic peptide which competitively blocks adhesion of S. mutans or other caries-causing bacteria to the tooth surface. Administration of such non-immunogenic peptides can be used to competitively inhibit bacterial adhesion, while avoiding implicating the immune system of the host. In related embodiments, the invention provides oral compositions for periodic topical application comprising such substances and an orally acceptable excipient.

The present invention also provides a recombinant polypeptide having an amino acid sequence corresponding to the amino acid sequence of SEQ. ID NO. 6 or 8, or an active fragment thereof preferably having murein hydrolase activity and/or a molecular weight of at least about 0.5 kDa.

In still another aspect, the invention provides an isolated DNA sequence encoding (i) a protein having the amino acid sequence of SEQ. ID NO. 6 or 8 or (ii) a polypeptide fragment of such a protein preferably having murein hydrolase activity and/or a molecular weight of at least about 0.5 kDa. Also provided is a vector comprising such a DNA sequence in operable association with a promoter. As well, host cells having introduced DNA including such a DNA sequence in operable association with a promoter are provided, and can for example be cultured to produce the protein or a fragment thereof.

The invention provides in another embodiment a method for lysing bacteria. In accordance with this inventive method, the bacteria are contacted with a protein from S. mutans or a polypeptide fragment or functional analog thereof, having murein hydrolase activity against the bacteria. The protein or polypeptide can include an amino acid sequence of SEQ. ID NO. 6 or 8, or an amino acid sequence sufficiently duplicative thereof to exhibit murein hydrolase activity.

The present invention provides novel methods and compositions for promoting oral health in a host animal, including a human. The invention also provides novel isolated DNA sequences encoding polypeptides, constructs including such DNA sequences, recombinant polypeptides, and related compositions and articles of manufacture. Additional objects, features and advantages of the invention will be apparent from the following description.

### DESCRIPTION OF THE FIGURES

FIG. 1A provides an amino acid sequence comparison of the 40 kDa predicted protein from S. mutans OMZ175 serotype f ORF and the 65 kDa predicted protein from S. mutans UA159 serotype c ORF. Dashed underline=serotype f sequence homologous to the sequence from the 65 kDa S. mutans A32-2 surface protein determined by CnBr protein sequencing (results shown in bold). Single underline=putative signal peptide and membrane spanning domains. Bold underlined=short repeat region. Double underlined=long repeat region. (*)=exact match, (.)=weakly similar, (:)=strongly similar.

FIG. 1B shows the genomic organization of S. mutans UA159 serotype c vs. OMZ175 serotype f. The UA159 ORF (SMU.609) (D. Ajdic, et al., Proc Natl Acad Sci USA, 22:14434-9 (2002) homologous to the 65 kDa A32-2 surface protein encodes a protein with a predicted molecular weight of 67 kDa and is located upstream of the cell surface protein antigen I/II. ∘=homology determined from CnBr protein sequencing. [symbol] =homologous coding region. [symbol] =coding sequence unique to serotype c. [symbol] =coding sequence unique to serotype f.

FIG. 2 provides a comparison of repeat regions. A. Short repeats (46 aa), three from S. mutans c 65 kDa protein (65-1s, 65-2s, and 65-3s), one from S. mutans f 40 kDa protein (40-1s), and four from S. agalactiae 60 kDa murein hydrolase protein (Bsp-1s, Bsp-2s, Bsp-3s, and Bsp-4s), compared to the consensus sequence for the SH3b repeat motif. B. Long repeats (88 aa) of S. mutans serotype c 65 kDa protein (65-1L, and 65-2L) compared to the single homologous sequence from S. agalactiae 60 kDa protein (Bsp-L). (*)=exact match, (.)=weakly similar, (:)=strongly similar.

FIG. 3 provides a comparison of the homologous DNA sequences from S. mutans UA159 serotype c (1) and OMZ175 serotype f (23). c: ORF SMU.609, encoding serotype c 67 kDa protein. The coding region for the serotype c ORF is from nt 1-1836. f: ORF encoding serotype f 40 kDa. The coding region for the serotype f ORF is from nt. 96-1103. The site of a frameshift mutation in the serotype f sequence at nt. 1016 and the subsequent stop codon for serotype f sequence (nt. 1101-1103) are underlined. * indicates mismatches in DNA sequence.

FIG. 4 is a digital image of a commassie stained SDS-PAGE gel of S. mutans surface proteins. Lane 1, serotype f; lane 3, serotype c; lanes 2 & 4, molecular weight markers (size in kDa indicated on right). * indicates positions of the 40 kDa and 65 kDa proteins.

FIG. 5 is a digital image of an immunoblot of S. mutans surface proteins probed with rat anti-S. mutans serotype c surface proteins. Lanes 1 & 4, molecular weight markers (size in kDa indicated on right); lane 2, OMZ175 (serotype f) surface proteins; lane 3, A32-2 (serotype c) surface proteins, lane 5, NG8 (serotype c) surface proteins. * indicates positions of the 40 kDa and 65 kDa proteins.

FIGS. 6A and 6B provide digital images of zymogram activity gels. For FIG. 6A, S. mutans peptidoglycan from serotype f (lanes 1-2) or serotype c (A32-2, lanes 3-7), S. sobrinus (lane 8), S. gordonii (lane 9), and S. oralis (lane 10), was incorporated into a 10% SDS-PAGE gel. Non-denatured surface proteins were separated on the gel: lane 1=A32-2, lane 2=OMZ175, lane M=Molecular weight markers, lane 3=A32-2, lane 4=NG8, lane 6=OMZ175, lane 7=UA159 (serotype c), and lanes 8-10=A32-2, as well as a purified SrtA enzyme preparation, lane 5. Zones of activity appear clear in the opaque gel. * indicates 65 kDa. For FIG. 6B, peptidoglycan from Actinobacillus actinomycetemcomitans 29522 was incorporated in to a similar gel, and non denatured surface proteins were from NG8 and A32-2 were separated on the gel along with molecular weight markers. Again, zones of activity appear clear in the opaque gel.

FIG. 6C provides bar graphs showing the results of biofilm adherence assays demonstrating that S. mutans serotype c surface protein preparations containing the 65 kDa protein inhibited the adherence of S. gordonii and S. oralis to microtiter wells, whereas the surface protein preparation from S. mutans OMA175 (serotype f) did not. * indicates a significant difference from control (no enzyme) using a paired t-test.

FIG. 7 shows binding of S. mutans fimbriae to whole saliva-coated ELISA plates. The ability of S. mutans fimbriae (0.33-33.00, μg/ml) to bind to saliva (undiluted and 1:2 and 1:10 dilutions) was determined by ELISA. The negative controls were wells that did not contain fimbriae. The ELISA absorbances (mean+SEM) represent a relative measurement of binding between fimbriae and saliva. ND=not determined.

FIG. 8 shows representative immunoblot of whole saliva from seven different subjects. Blots were probed with fimbriae from S. mutans A32-2, followed by rat antibody to enriched fimbriae of S. mutans A32-2 and alkaline phosphatase-labeled goat antibody to rat IgG. Whole salivas from seven subjects (lanes 1-7) are shown. Arrow on right indicates molecular weight of the major salivary component (52 kDa) that bound fimbriae.

FIG. 9 shows representative coomassie brilliant blue and silver dual stained SDS-PAGE gel of purified salivary protein. Purified salivary protein was collected using preparative gel electrophoresis and analyzed using SDS-PAGE. The arrow on the right indicates the molecular weight of the isolated salivary component (52 kDa).

FIG. 10 shows S. mutans A32-2 fimbriae binding to salivary proteins. ELISA plate wells were coated with the purified salivary protein (65.0, μg/ml), whole saliva (diluted 1:10) and amylase (10.0, μg/ml). After blocking with 1% BSA, the S. mutans A32-2 fimbriae preparation (33.0 μg/ml) was incubated with the various salivary proteins. The controls (open bars) did not include fimbriae. The ELISA absorbances (mean±SEM) represent a relative measurement of binding between fimbriae and salivary components.

FIG. 11 shows neutralization of S. mutans fimbriae binding to saliva-coated surfaces by a purified salivary protein. The S. mutans A32-2 fimbriae preparation (33.0 μg/ml) was incubated with varying concentrations (0.5-65.0 μg/ml) of the purified salivary protein and assayed for binding to saliva.

FIG. 12 provides a representative anti-amylase immunoblot. Purified salivary protein (65.0, μg/ml), undiluted whole saliva and human α-amylase (10.0, μg/ml) probed with rabbit antibody to human α-amylase. Human whole saliva (lane 1); human α-amylase (lane 2); and purified salivary protein (lane 3) are shown. The arrow on the right indicates a molecular weight of 52 kDa.

### DETAILED DESCRIPTION

For the purpose of promoting an understanding of the principles of the invention, reference will now be made to certain preferred embodiments thereof and specific language will be used to describe the same. It will nevertheless be understood that no limitation of the scope of the invention is thereby intended, such alterations, further modifications and applications of the principles of the invention as described herein being contemplated as would normally occur to one skilled in the art to which the invention relates.

The present invention features, in certain aspects, newly characterized murein hydrolase enzyme activity of S. mutans, related recombinantly-produced proteins and protein fragments, and related methods and compositions useful in the promotion of oral health. Certain forms of the invention provide methods for promoting oral health involving the use of murein hydrolase polypeptides active against bacteria in the plaque. Other forms of the invention relate to oral compositions including the murein hydrolase polypeptide, and oral care products including such compositions. Methods and compositions of the invention may be beneficial, for example, in controlling dental plaque and/or in controlling dental caries in a host. In still other aspects, the invention provides novel recombinant proteins and recombinant polypeptide fragments thereof, and isolated DNA sequences, vectors, and host cells suitable for the production of such recombinant proteins and fragments.

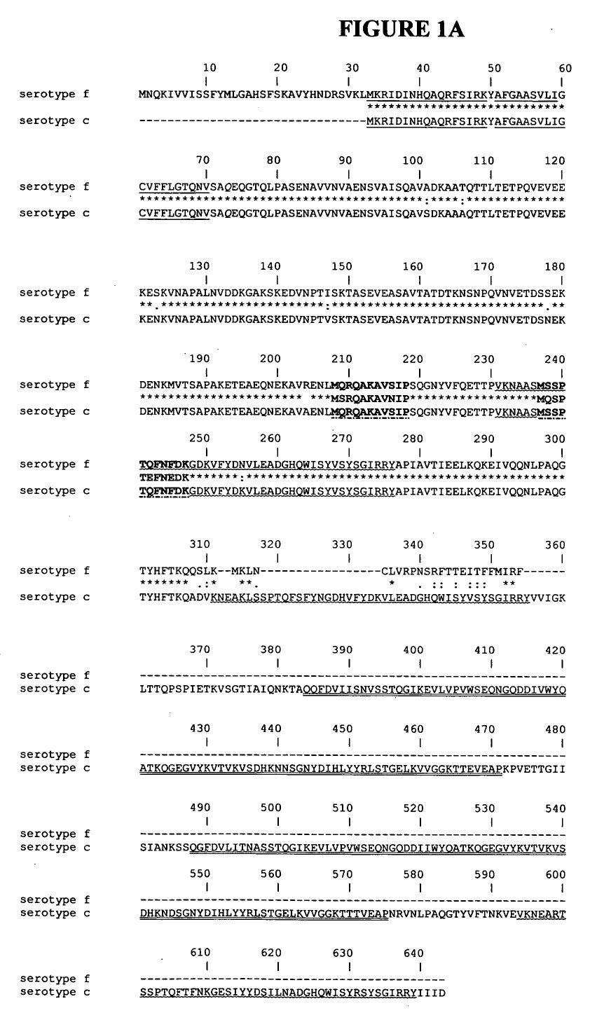

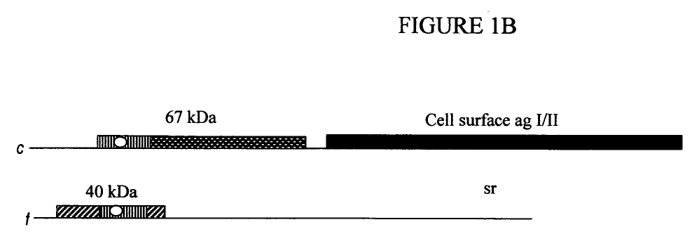

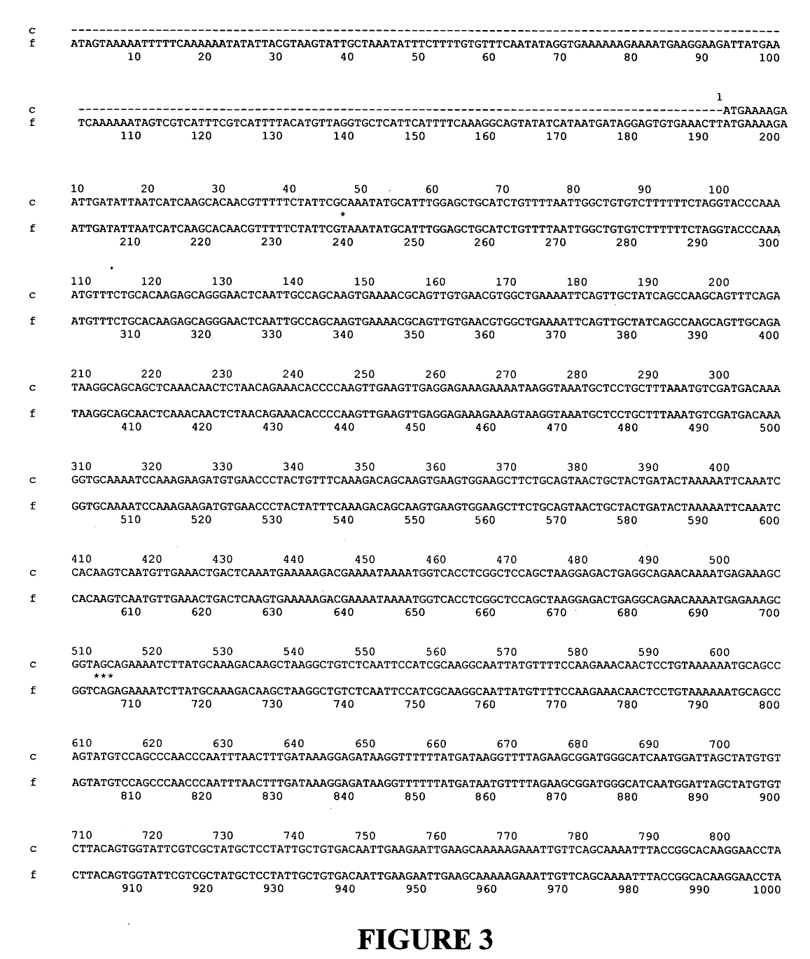

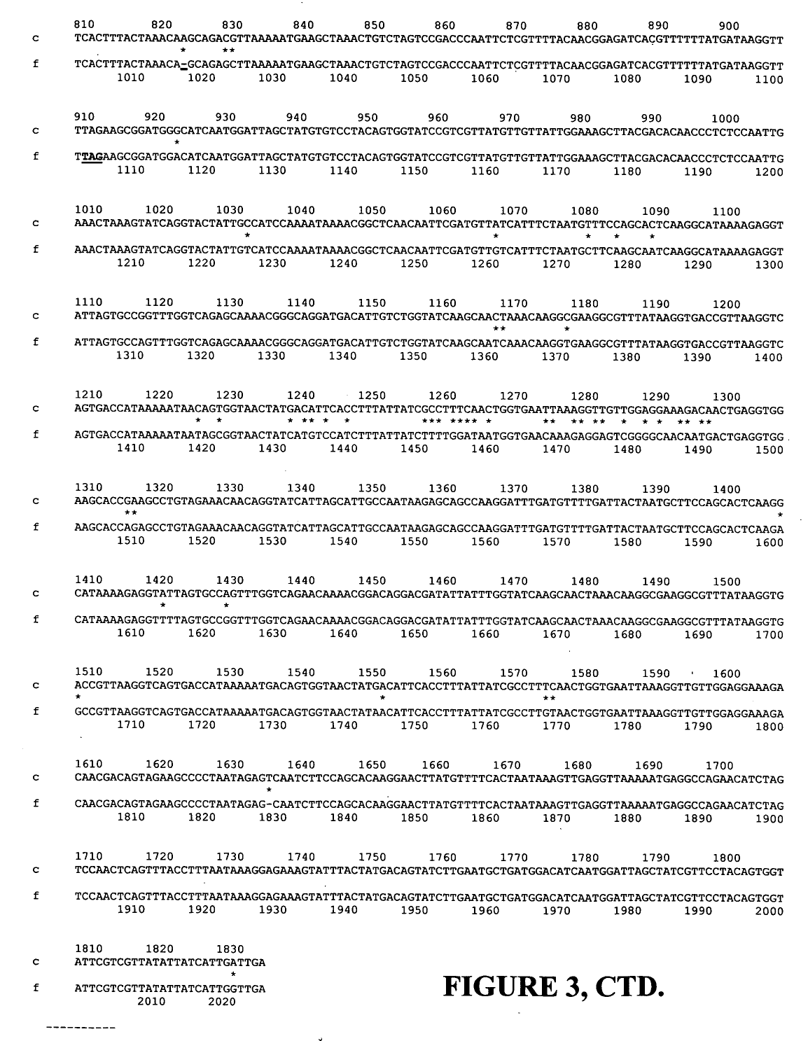

Turning now to a more detailed discussion of the invention, the S. mutans protein having murein hydrolase activity was initially isolated in surface preparations of S. mutans, serotype c, and characterized to have a molecular weight of about 65 kilodaltons (kDa). The protein was purified and portions were sequenced, with a first experimental set resulting in the identification of the sequences MSSQAKANNIP (SEQ. I.D. NO. 1), MQRPTEFXEDK (SEQ. I.D. NO. 2), and a second experimental set resulting in the identification of sequences MSRQAKAVNIP (SEQ. I.D. NO. 3) and MQSPTEFNEDK (SEQ. I.D. NO. 4). These sequences were thus expected to be internal sequences in the protein or at least bear substantial homology to internal sequences in the protein. Amino acid sequence comparison demonstrated that these sequences exhibit substantial identity to sequences within a 40 kDa S. mutans putative cell wall protein precursor (strain OMZ15, serotype f) reported by J. A. Ogier et al., Infection and Immunity, May 1991, pp. 1620-1626 (see SEQ. I.D. NO. 6, and genbank accession no. A60328). The coding nucleotide sequence for such protein is shown in SEQ. I.D. NO. 5, from nucleotides 96 to 1103. Sequences similar to those shown in SEQ. I.D. NO. 6 and SEQ. I.D. NO. 5 were thus expected within the identified 65 kDa S. mutans protein and the DNA which encodes it, respectively. A search of the S. mutans UA159 serotype c genome resulted in the identification of an open reading frame and its predicted translation product as shown in FIGS. 3 and 1A, and SEQ. ID NOS. 7 and 8, respectively.

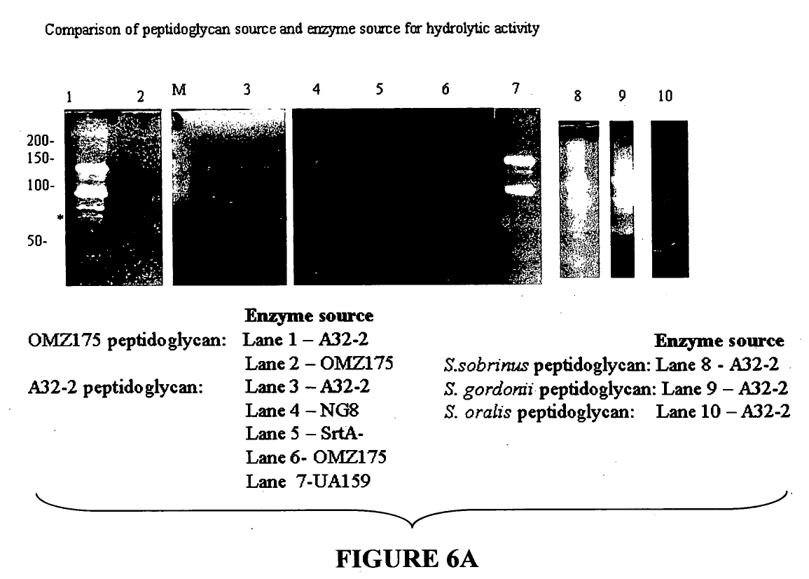

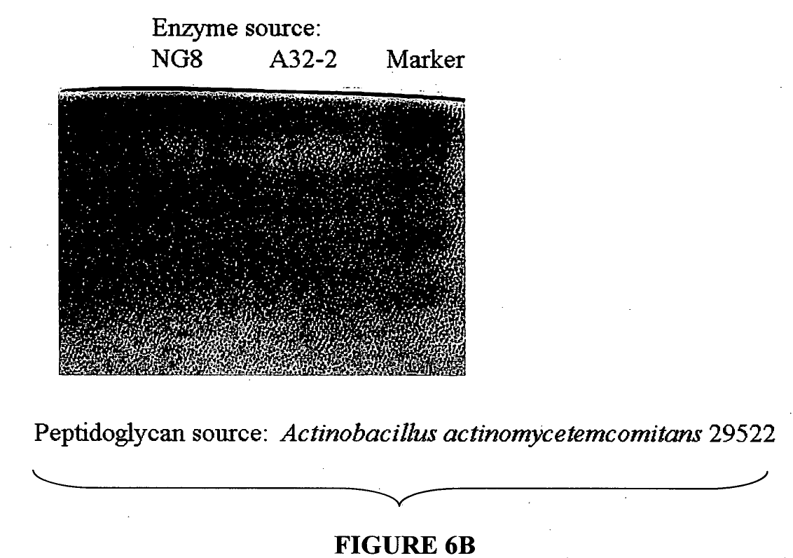

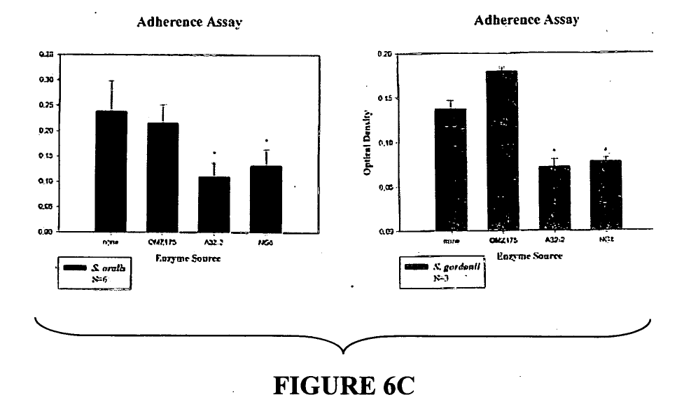

In further work, zymogram activity gel analysis has shown that the purified S. mutans 65 kDa protein possesses both autolytic murein hydrolase activity and activities against a number of Streptococcus and other bacteria that commonly occur in dental plaque (see FIGS. 6A, 6B, 6C, and 6D). In particular, in testing to date, the protein has demonstrated hydrolase activity against species of Streptococcus parasanguis (FW213), Streptococcus oralis (10557), Streptococcus sanguis (10556), Streptococcus gordonii (10558), Streptococcus sobrinus (27607), and Actinobacillus actinomycetemcomitans (29522).

Biofilm adherence assays have also been conducted, in which Streptococcus oralis and Streptococcus gordonii bacteria were incubated in microtiter wells in the presence and absence of an S. mutans serotype c surface protein preparation containing the murein hydrolase protein. Adherence of these bacteria was significantly inhibited in the presence of the surface protein preparation, as shown in FIG. 6F.

It has thus been evidenced that the S. mutans surface protein has murein hydrolase activity effective against Streptococcus and other bacterial species common to dental plaque. This protein, as well as fragments and analogs thereof having murein hydrolase activity, can thus be used in the treatment and lysis of bacteria occurring on or within a host, including within dental plaque or other surfaces in the oral cavity. Accordingly, in one aspect of the invention, such polypeptides having murein hydrolase activity against bacteria occurring in dental plaque or other deposits upon teeth or other oral surfaces, are used orally in treatments for promoting oral health.

In these regards, dental plaque is a soft deposit that accumulates on the teeth and contains characteristic bacterial cells. The cells are contained within a matrix containing proteins, polysaccharides and lipids formed from bacterial products and saliva. Inorganic components can also be found in dental plaque, such as calcium and phosphorus. Dental calculus is characterized by a substantial increase in inorganic components and the calcification of dental plaque. Calculus deposits are significantly more difficult to remove, and provide surfaces that are readily colonized by plaque.

The development of dental plaque has been studied in humans and other animal model systems. A newly cleaned tooth surface is rapidly covered with a glycoprotein deposit derived from salivary constituents and referred to as the “pellicle”. The pellicle includes albumin, lysozyme, amylase, immunoglobulin A, proline-rich proteins and mucins. The pellicle-coated tooth surface is first colonized by Gram-positive bacteria such as Streptococcus sanguis, Streptococcus parasanguis, and Actinomyces viscosus. These organisms are “primary colonizers” of dental plaque. Bacterial surface molecules interact with components of the dental pellicle to enable the bacteria to attach or adhere to the pellicle-coated tooth surface. After the initial colonization of the tooth surface, plaque increases not only by multiplication of bacteria already attached to the tooth surface, but also by subsequent attachment and multiplication of new bacterial species to cells of bacteria already present in the plaque mass. Secondary colonizers include the gram-positive species, Streptococcus mutans (considered to be the major pathogenic agent causing dental caries), and Gram-negative species such as Fusobacterium nucleatum, Prevotella intermedia, and Capnocytophaga species, which adhere to Gram-positive species already present in the existing plaque mass. After about a week of plaque accumulation, other Gram-negative species may also be present in plaque. These species represent what is considered to be the “tertiary colonizers”, and include Porphyromonas gingivalis, Campylobacter rectus, Eikenella corrodens, Actinobacillus actinomycetemcomitans, and the oral spirochetes (Treponema species).

The protein or other polypeptide having murein hydrolase activity can be delivered to the oral cavity in any suitable oral formulation on a regular basis, as needed to beneficially reduce colonization of tooth or other surfaces by bacteria against which the murein hydrolase polypeptide is active, e.g. Gram-positive bacteria such as Streptococcus sanguis, Streptococcus mutans, and/or Actinomyces viscosus, or other bacteria which may be primary, secondary or tertiary colonizers of dental plaque. Such compositions can be packaged and marketed for improving oral hygiene by eliminating or inhibiting bacteria within the oral cavity, e.g. by eliminating or inhibiting bacteria involved in the formation or persistence of dental plaque, and/or simply for reducing the occurrence of dental plaque or other oral conditions associated with the subject bacteria. For these purposes, packaging for the oral compositions may contain or otherwise be associated with written indicia relating to use of the compositions for these purposes.

The murein hydrolase proteins or fragments thereof can be used in active immunization protocols and/or to raise antibodies which may be used, for example, in passive immunization protocols to inhibit caries formation in mammals and other animals. For example, the isolated S. mutans protein has been used to raise rat antibodies to the protein, which have proven to react with all 20 strains of S. mutans tested to date, and to show lower or no significant reactivity to other oral bacteria.

The inhibition of activities of the S. mutans murein hydrolase enzyme may be used to reduce or eliminate the lytic activity of S. mutans bacteria existing in dental plaque or other areas of the oral cavity, and thus modulate the bacterial colonies of the oral cavity and the characteristics of dental plaque. For example, monoclonal or polyclonal antibodies to the murein hydrolase may be administered orally in amounts effective to inhibit the lytic activity of S. mutans bacteria. In addition or alternatively, one or more substrates for the murein hydrolase enzyme may be administered to the oral cavity in order to competitively and/or permanently (e.g. as in so-called “suicide substrates”) inhibit the lytic activity of S. mutans therein. Illustratively, bacterial cell wall components such as peptidoglycans that are targeted by the enzyme, or fragments thereof, may serve as such substrates. As well, the murein hydrolase protein can be used in screens for other agents such as small molecular pharmaceutical agents that are effective and can be used to inhibit the protein. Still further, preliminary data provides evidence that the protein has some capacity to bind amylase, which may play a role in the binding of S. mutans to tooth surfaces. Amylase-binding protein or fragments may be used in such circumstance to competitively inhibit binding or other related colonization activities of S. mutans. These and other variations will be understood by and available to those of ordinary skill in the art given the teachings herein.

The present invention also provides recombinantly produced polypeptides and genetic materials and methods useful in making such polypeptides. The DNA of the identified 65 kDa S. mutans protein can, for example, be operably associated with a promoter and incorporated in a suitable vector, e.g. a transformation vector, and used to transform cells in vitro or in vivo. In one aspect, microorganisms can be transformed using these inventive DNA sequences and caused to express a polypeptide in vitro. For example, using methods well known in the relevant art, microorganisms, including but not limited to bacteria such as E. coli cells, may be transformed such that they synthesize inventive proteins in relatively large amounts. Unicellular hosts are selected by consideration of their compatibility with the chosen vector, the toxicity of the product coded on expression by the DNA sequences of this invention to them, their secretion characteristics, their ability to fold proteins correctly, their stability and culturing requirements, and the ease of purification of the products coded on expression by the DNA sequences of this invention. Given these parameters, skilled practitioners will be able to select a variety of suitable hosts for use in this aspect of the invention. In other aspects, such vectors or other DNA constructs can be used to transform a host animal in vivo to express a polypeptide, for example an antigenic polypeptide in the case of a DNA vaccine.

The present invention is not intended to be limited by the choice of vector or host cell. It should of course be understood that not all vectors and expression control sequences will function equally well to express the DNA sequences of this invention. Neither will all hosts function equally well with the same expression system. However, one of skill in the art may make a selection among these vectors, expression control sequences, and hosts without undue experimentation and without departing from the scope of this invention.

Similarly, it will be understood that it is oftentimes convenient or even preferred that recombinant or synthetic production yields a protein modified from its native form, that is, a polypeptide that differs as to its structure from the naturally occurring polypeptide, but which retains the biological activity of the naturally occurring polypeptide. For example, a modified polypeptide may contain one or more additional amino acids, at one or both ends of the polypeptide chain, may include one or more point mutations, deletions, truncations, or the like. Polypeptides incorporating such modifications while exhibiting the murein hydrolase or other beneficial properties of the native 65 kDa S. mutans polypeptide are contemplated as being within the present invention. As well, it will be understood that additional polynucleotides encoding proteins functionally analogous to the 65 kDa S. mutans protein identified herein may be obtained, for example, by generating DNA probes utilizing the sequences disclosed herein, and screening appropriate S. mutans DNA libraries using techniques within the purview of those practiced in this area.

Preferred aspects of the present invention involve uses as described herein of proteins or fragments thereof (desirably at least 0.5 kDa) which have an amino acid sequence having at least about 70% identity to an amino acid sequence of SEQ. I.D. NO. 6 or SEQ. I.D. NO. 8, more preferably at least about 80% identity, and most preferably at least about 90% identity. Other preferred aspects of the invention involve uses as described herein of nucleic acid molecules which have a nucleotide sequence encoding a protein or fragment thereof, which nucleotide sequence has at least about 70% identity to a nucleotide sequence of SEQ. I.D. NO. 5 from nucleotide 816 to 1820, or to a nucleotide sequence of SEQ. I.D. NO. 7 from nucleotides 115 to 1833, more preferably at least about 80% identity, and most preferably at least about 90% identity. Such nucleotides sequences preferably code for a polypeptide of at least about 0.5 kDa. Such proteins or fragments desirably have, and such nucleotide sequences desirably code for proteins or fragments that have, murein hydrolase and/or another beneficial activity of the native 65 kDa S. mutans protein.

Percent identity, as used herein, is intended to mean percent identity as determined by comparing sequence information using the advanced BLAST computer program, version 2.0.8, available from the National Institutes of Health, USA. The BLAST program is based on the alignment method of Karlin and Altschul, Proc. Natl. Acad. Sci. USA 87: 2264-68 (1990) and as discussed in Altschul, et al., J. Mol. Biol. 215: 403-10 (1990); Karlin and Altschul, Proc. Natl. Acad. Sci. USA 90: 5873-7 (1993); and Altschul et al. (1997) Nucleic Acids Res. 25: 3389-3402. Briefly, the BLAST program defines identity as the number of identical aligned symbols (i.e., nucleotides or amino acids), divided by the total number of symbols in the shorter of the two sequences. The program may be used to determine percent identity over the entire length of the proteins being compared.

Preferred default parameters for the BLAST program, blastp, include: (1) description of 500; (2) Expect value of 10; (3) Karlin-Altschul parameter λ=0.270; (4) Karlin-Altschul parameter K=0.0470; (5) gap penalties: Existence 11, Extension 1; (6) H value=4.94e⁻³²⁴, (6) scores for matched and mismatched amino acids found in the BLOSUM62 matrix as described in Henikoff, S. and Henikoff, J. G., Proc. Natl. Acad. Sci. USA 89:10915-10919 (1992); Perarson, W. R., Prot. Sci. 4:1145-1160 (1995); and Henikoff, S and Henikoff, J. G., Proteins 17:49-61 (1993). The program also uses an SEG filter to mask-off segments of the query sequence as determined by the SEG program of Wootton and Federhen Computers and Chemistry 17:149-163, (1993).

Additional information concerning the identified 65 kDa protein can be found in Ray et al, Interactions of Streptococcus mutans Fimbrial-Associated Surface Proteins with Salivary Components, Clin. Diag Lab. Immunol., May 1999, 6 (3) pp. 400-4, and M. Fontana et al., Intranasal Immunization Against Dental Caries with Streptococcus mutans Enriched Fimbrial Preparation, Clin. Diag. Lab. Immunol., May 1999, 6 (3) pp. 405-9. The protein, previously termed “SmaA”, was preliminarily identified as an amylase-binding protein, believed to be an adhesin of S. mutans functioning to mediate attachment of the bacteria to the salivary pellicle. It will be understood that the applicants do not intend their inventions disclosed herein to be bound to these prior-disclosed aspects unless expressly stated as such.

In accordance with methods of the invention, the 65 kDa S. mutans protein, or an active peptide derived from this protein, may be administered in an oral composition such as a dentifrice (e.g. toothpaste), a mouthwash, a chewing gum, or the like. Various conventional excipients and additives for such oral compositions, and their relative amounts, are well known. These are reviewed for example in U.S. Pat. No. 5,500,206, portions of which are summarized and incorporated in the discussions below for ease of reference and in order to facilitate a description of certain components of oral compositions of the present invention.

In general, the oral compositions of the invention include an effective amount of the 65 kDa protein or an active fragment thereof in a suitable oral carrier. The protein or active fragment will typically be included at a level of about 0.01% to about 5% by weight of the oral composition, more preferably about 0.1% to about 2% by weight, and most preferably about 0.2 to about 1% by weight.

The oral carrier employed may comprise, for example, a solid or liquid filler, and diluents suitable for use in oral compositions intended for contact with the oral tissues of humans and lower animals. Such compositions include for example mouthwashes, mouth rinses, mouth sprays, dental treatment solutions, toothpastes, dental gels, tooth powders, prophylaxis pastes, lozenges, chewing gums and the like. Dentifrices, mouthwashes and chewing gums represent preferred compositions.

The topical, oral carriers of the present invention comprise components typically used in such compositions which are well known to a skilled practitioner. Such components include, but are not limited to, anticaries agents, antiplaque agents, anticalculus agents, dental abrasives, surfactants, flavoring agents, sweetening agents, binders, humectants, thickening agents, buffering agents, preservatives, coloring agents and pigments, ethanol and water.

Water may be used in the topical, oral carriers of the compositions of the present invention, and when used will usually be of low ion content and free of organic impurities. Water may comprise from about 2% to about 99%, more typically from about 20% to about 95% of the compositions of the present invention. When in the form of toothpaste, the compositions preferably comprise from about 2% to about 45%, more preferably from about 30% to about 40%, water, while mouthwashes comprise preferably from about 45% to about 95%, more preferably from about 75% to about 90%, water.

A variety of dental abrasives will be useful in the topical, oral carriers of the compositions of the present invention. The material selected must be one which is compatible with the composition of interest and does not excessively abrade dentine. These include, for example, silicas, including gels and precipitates, calcium carbonate, dicalcium orthophosphate dihydrate, calcium pyrophosphate, tricalcium phosphate, calcium polymetaphosphate, insoluble sodium polymetaphosphate, hydrated alumina, and resinous abrasive materials such as particulate condensation products of urea and formaldehyde, and other materials such as those disclosed by Cooley et al. in U.S. Pat. No. 3,070,510, issued Dec. 25, 1962. Mixtures of abrasives may also be used.

The silica abrasive polishing materials useful herein, as well as the other abrasives, generally have an average particle size ranging between about 0.1 and 30 microns, preferably between about 5 and 15 microns. The silica abrasive can be precipitated silica or silica gels such as the silica xerogels described in U.S. Pat. No. 3,538,230, issued Mar. 2, 1970 to Pader et al., and in U.S. Pat. No. 3,862,307, issued Jun. 21, 1975 to DiGiulio. Preferred are the silica xerogels marketed under the tradename Syloid. RTM. by the W. R. Grace & Company, Davidson Chemical Division.

Preferred precipitated silica materials include those marketed by the J. M. Huber Corporation under the tradename, Zeodent. RTM., particularly the silica carrying the designation Zeodent 119. RTM. These silica abrasives are described in U.S. Pat. No. 4,340,583, Wason, issued Jul. 20, 1982, incorporated herein by reference. Other suitable abrasives include alumina and the insoluble metaphosphates such as insoluble sodium metaphosphate (IMP).

Mixtures of abrasives may be used. The total amount of abrasive in the dentifrice embodiments of this invention can range from about 6% to about 70%, preferably from about 15% to about 50%, when the dentifrice is a toothpaste. Higher levels, as high as 90%, may be used if the composition is a tooth powder.

Flavoring agents can also be added to the oral compositions of the present invention. Suitable flavoring agents include menthol, oil of wintergreen, oil of peppermint, oil of spearmint, oil of sassafras, and oil of clove. Flavoring agents are generally included in the subject compositions in amounts of from about 0% to about 3%, preferably from about 0.04% to about 2% by weight.

Coloring agents may be added to compositions of the present invention to improve appearance. If present, coloring agents typically are included at levels of from about 0.001% to about 0.5% by weight.

Sweetening agents may also be used in the compositions of the present invention. Sweetening agents which can be used include aspartame, acesulfame, saccharin salts, dextrose, levulose thaumatin, Dtryptophan, dihydrochalcones, and cyclamate salts. Saccharin salts are preferred. Sweetening agents are generally used in the subject compositions in amounts of from about 0% to about 6%, preferably from about 0.005% to about 5% by weight.

Oral compositions can also contain a surfactant. Suitable surfactants are those which are reasonably stable and form suds throughout a wide pH range, including nonsoap anionic, nonionic, cationic, zwitterionic and amphoteric organic synthetic detergents, and compatible mixtures thereof.

Many of these suitable surfactants are disclosed in U.S. Pat. No. 4,051,234, issued to Gieske et al. on Sep. 27, 1977, and in U.S. Pat. No. 3,959,458 issued to Agricola, Briner, Granger and Widder on May 25, 1976. Surfactants are typically present in compositions of the present invention at a level of from 0% to about 10%, preferably from about 0.2% to about 4% by weight. Surfactants may also be used as solubilizing agents to help retain sparingly soluble components, e.g., some flavoring agents, in solutions. Surfactants suitable for this purpose include polysorbates and poloxamers.

In preparing oral compositions of the present invention, it is often desirable to add binders and/or thickening agents, particularly to toothpaste compositions. Preferred binders and thickening agents include for example, carboxyvinyl polymers, polysaccharide gums such as xanthan gum, carrageenan, hydroxyethyl cellulose and water soluble salts of cellulose ethers such as sodium carboxymethyl cellulose and sodium carboxymethyl hydroxyethyl cellulose. Natural gums such as gum karaya, gum arabic, and gum tragacanth can also be used. Colloidal magnesium aluminum silicate or finely divided silica can be used as part of the thickening agent to further improve texture. These binders and thickening agents are generally present in the compositions of the present invention in amounts of from about 0% to about 6%, preferably from about 0.1% to about 5% by weight.

Another optional component of the oral carriers of the compositions of the present invention is a humectant. The humectant serves to keep toothpaste compositions from hardening upon exposure to air, and to give mouthwash and toothpaste compositions a moist feel to the mouth. Certain humectants can also impart desirable sweetness of flavor to mouthwash and toothpaste compositions. The humectant, on a pure humectant basis, generally comprises from about 0% to about 70%, preferably from about 2% to about 55%, by weight of the compositions herein. Suitable humectants for use in compositions of the present invention include edible polyhydric alcohols such as glycerin, sorbitol, xylitol, polyethylene glycol, and propylene glycol, especially sorbitol and glycerin.

Opacifiers may also be used in toothpastes of the present invention to render the toothpaste opaque. Suitable opacifiers include titanium dioxide and some abrasives including, for example, magnesium aluminum silicate. Opacifiers generally comprise from about 0% to about 4%, preferably from about 0.5% to about 3% by weight of the compositions herein.

Other optional components of the compositions of the present invention are preservatives. The preservatives prevent microbial growth in the compositions. Suitable preservatives include methylparaben, propylparaben, benzoates and ethanol. If the preservative is ethanol, it generally comprises from 0% to about 35% by weight, preferably from about 5% to about 15%, of the compositions herein. Other preservatives generally comprise from about 0% to about 5% by weight, preferably from about 0.1% to about 2%, of the compositions herein.

Antimicrobial, antiplaque agents can also optionally be present in the oral compositions of the present invention, on the condition that they are compatible with the 65 kDa protein or fragment. Such agents may include, but are not limited to, triclosan, 2,4,4′-trichloro-2′-hydroxydiphenyl ether, as described The Merck Index, 11th Ed. (1989), p. 1520 (entry No. 9573); in U.S. Pat. No. 3,506,720; and in Eur. Pat. Appl. No. 0,251,591 of Beecham Group, PLC, published Jan. 7, 1988, chlorhexidine, (Merck Index, No. 2090), alexidine (Merck Index, No. 222); hexetidine (Merck Index, No. 4624); sanguinarine (Merck Index, No. 8320); benzalkonium chloride (Merck Index, No. 1066); salicylanilide (Merck Index, No. 8299); domiphen bromide (Merck Index, No. 3411); cetylpyridinium chloride, (CPC) (Merck Index, No. 2024); tetradecylpyridinium chloride, (TPC); N-tetradecyl-4ethylpyridinium chloride (TDEPC); octenidine; delmopinol, octapinol, and other piperidino derivatives; nicin preparations; zinc/stannous ion agents; antibiotics such as augmentin, amoxicillin, tetracycline, doxycycline, minocycline, and metronidazole; and peroxides, such as cylium peroxide, hydrogen peroxide, and magnesium monoperthalate and its analogs as described in U.S. Pat. No. 4,670,252; and analogs and salts of the above antimicrobial antiplaque agents. If present, the antimicrobial antiplaque agents may comprise from about 0% to about 6%, preferably from about 0.1% to about 5% by weight of the compositions of the present invention.

Anti-inflammatory agents can also be present in the oral compositions of the present invention. Such agents may include, but are not limited to, non-steroidal anti-inflammatory agents such as ketorolac, flurbiprofen, ibuprofen, naproxen, indomethacin, aspirin, ketoprofen, piroxicam and meclofenamic acid. If present, the anti-inflammatory agents generally comprise from about 0.001% to about 5% by weight of the compositions of the present invention.

Nutrients can also be present in the oral composition of the present invention, on condition that they are compatible with the 65 kDa protein or fragment. Such agents may include folate, retinoids (Vitamin A), Vitamin C, Vitamin E and zinc. If present, the nutrients generally comprise from about 0.001% to about 10% by weight of the compositions of the present invention.

Other optional ingredients include a safe and effective amount of a fluoride ion source, which typically is in the form of a water-soluble fluoride compound. This water-soluble fluoride compound is typically present in the compositions of the present invention in an amount sufficient to give a fluoride concentration of from about 0.0025% to about 5.0% by weight, preferably from about 0.005% to about 2.0% by weight. Preferred fluoride sources are sodium fluoride, acidulated phosphate fluoride, and sodium monofluorophosphate. U.S. Pat. No. 3,678,154, issued Jul. 18, 1972 to Widder et al., the disclosure of which is incorporated herein by reference, discloses such salts as well as others.

Compositions of the present invention may also include one or more anticalculus agents, on the condition that they are compatible with the 65 kDa protein or fragment. Anticalculus agents which may be useful in the compositions of the present invention include diphosphonates such as 1azocycloheptane-2,2-diphosphonate AHP) and ethane-1-hydroxy-1,1-diphosphonate (EHDP), sodium zinc citrate, phosphocitrate, tripolyphosphate, and linear polycarboxylate (LPC); pyrophosphates or polyphosphates such as those disclosed in U.S. Pat. No. 4,590,066 issued to Parran & Sakkab on May 20, 1986 (e.g. tetrasodium pyrophosphate, tetrapotassium pyrophosphate, and dihdrogen disodium pyrophosphate); polyacrylates and other polycarboxylates such as those disclosed in U.S. Pat. No. 3,429,963 issued to Shedlovsky on Feb. 25, 1969 and U.S. Pat. No. 4,304,766 issued to Chang on Dec. 8, 1981; and U.S. Pat. No. 4,661,341 issued to Benedict & Sunberg on Apr. 28, 1987; polyepoxysuccinates such as those disclosed in U.S. Pat. No. 4,846,650 issued to Benedict, Bush & Sunberg on Jul. 11, 1989; nitrilotriacetic acid and related compounds as disclosed in U.S. Pat. No. 3,678,154 issued to Widder & Briner on Jul. 18, 1972; polyphosphonates as disclosed in U.S. Pat. No. 3,737,533 issued to Francis on Jun. 5, 1973, U.S. Pat. No. 3,988,443 issued to Ploger, Schmidt-Dunker & Gloxhuber on Oct. 26, 1976 and U.S. Pat. No. 4,877,603 issued to Degenhardt & Kozikowski on Oct. 31, 1989; all of these patents are incorporated herein by reference. If present, the anticalculus agents generally comprise from about 0.2% to about 13%, preferably from about 0.4% to about 6% of the compositions of the present invention. Preferred anticalculus agents are pyrophosphate and AHP.

As indicated, compositions of the present invention may be in the form of toothpastes. Components of such toothpastes generally include a dental abrasive (from about 10% to about 50%), a surfactant (from about 0.5% to about 10%), a thickening agent (from about 0.1% to about 5%), a humectant (from about 10% to about 55%), a flavoring agent (from about 0.04% to about 2%), a sweetening agent (from about 0.1% to about 3%), a coloring agent (from about 0.01% to about 0.5%) and water (from about 2% to about 45%). Such toothpastes may also include one or more of an anticaries agent (from about 0.05% to about 0.3% as fluoride ion), an anticalculus agent (from about 0.1% to about 13%), and an antiplaque agent (from about 0.1% to about 5%).

Other compositions of the present invention are mouthwashes and mouth sprays. Components of such mouthwashes and mouth sprays include water (from about 45% to about 95%), ethanol (from about 0% to about 25%), a humectant (from about 0% to about 50%), a surfactant agent (from about 0.01% to about 7%), a flavoring agent (from about 0.04% to about 2%), a sweetening agent (from about 0.1% to about 3%), and a coloring agent (from about 0.001% to about 0.5%). Such mouthwashes and mouth sprays may also include one or more of an anticaries agent (from about 0.05% to about 0.3% as fluoride ion), an anticalculus agent (from about 0.1% to about 3%), and an antiplaque agent (from about 0.1% to about 5%).

Other compositions of the present invention are dental solutions. Components of such dental solutions generally include water (from about 90% to about 99%), preservative (from about 0.01% to about 0.5%), thickening agent (from about 0% to about 5%), flavoring agent (from about 0.04% to about 2%), sweetening agent (from about 0.1% to about 3%), and surfactant (from 0% to about 5%).

Other preferred compositions may be non-aqueous mouth rinses. Suitable components are disclosed in U.S. Pat. No. 4,312,889 issued Jan. 26, 1982 to Melsheimer, and in U.S. Pat. No. 5,143,720 issued Sep. 1, 1992 to Lopes, both incorporated herein by reference. Alcohol free mouth rinses are also preferred. Suitable compositional components can disclosed in U.S. Pat. No. 4,919,918, issued Apr. 24, 1990 to Cole et al., in U.S. Pat. No. 5,283,056, issued Feb. 1, 1994 to Chung et al., in U.S. Pat. No. 5,284,648, issued Feb. 8, 1994 to White et al., and in PCT Appl. No. 9 401 081, published Jan. 20, 1990.

Other embodiments of the oral compositions herein include lozenges. Suitable lozenge components (e.g. a candy base) are disclosed in U.S. Pat. No. 4,931,473, issued Jun. 5, 1990, to Kelleher et al., and in U.S. Pat. No. 4,139,627, issued Feb. 13, 1979 to Lane et al., and in PCT Appl. No. 9 401 081, of Konopa, published Jan. 20, 1994.

Other compositions include chewing gums. Chewing gum components (e.g. gum base, flavoring and sweetening agents) are disclosed in U.S. Pat. No. 4,083,955, issued Apr. 11, 1978 to Grabenstetter et al.

Still other oral compositions include foodstuffs containing typical components such as protein and/or carbohydrate sources, and chew toys or other chew articles for animals, e.g. incorporating rawhide or other collagenous matter.

The pH of the subject compositions and/or its pH in the mouth can be any pH which is safe for the mouth's hard and soft tissues. Such pH's are generally from about 3 to about 10, more preferably from about 4 to about 8.

When the oral composition is a toothpaste, typically from about 0.3 grams to about 15 grams, preferably from about 0.5 grams to about 5 grams, more preferably from about 1 to about 2 grams, of toothpaste is applied to an applicating device e.g., a toothbrush. The applicating device is then contacted with the oral cavity surfaces in a manner such that the oral composition is contacted with tissue of the oral cavity, especially the teeth and gums. The applicating device may be further used to effect an even distribution of the oral composition to the tooth surface, for example by brushing. The application preferably lasts for a period of from about 15 seconds to about 10 minutes, more preferably from about 1 minute to about 2 minutes. Following application, the toothpaste residue is typically removed from the tooth surface by using a liquid acceptable to the oral cavity, typically water, to rinse the oral cavity.

When the oral composition is a mouthwash, typically from about 1 ml to about 20 ml, preferably from about 2 ml to about 15 ml., most preferably from about 10 ml to about 15 ml, of liquid mouthwash containing the protein or active fragment is introduced to the oral cavity. The liquid mouthwash is then agitated for from about 10 seconds to about 30 min., preferably from about 15 seconds to about 3 min., more preferably from about 30 seconds to about 2 minutes, within the oral cavity to obtain an improved distribution of the mouthwash over the tissue of the oral cavity. Following agitation, the mouthwash is typically expectorated from the oral cavity. Application frequency is preferably from about once daily to about 4 times daily, more preferably from about 3 times weekly to about 3 times daily, more preferably still from about once to about twice daily. The period of such treatment typically ranges from about one day to a lifetime.

In one aspect of the invention, a polypeptide sequence representing a fragment or portion of the 65 kDa S. mutans protein may be used to competitively inhibit S. mutans. Such polypeptides can for example be derived from a recombinant or naturally-derived full protein, and identification and isolation of the competitive fragment. Alternatively, a DNA sequence encoding an amino acid sequence including the competitive domain of the protein can be utilized in the expression of a recombinant, competitive fragment. It is contemplated that such fragments will have a molecular weight of at least about 0.5 kDa, generally in the range of about 0.5 kDa up to about 20 kDa.

In one form of the invention, the competitive fragment will be of a size rendering it non-immunogenic to the host. As is known in the art, polypeptides having molecular weights less than about 10 kDa will usually not elicit an immune response, and thus fragments of less than about 10 kDa will desirably be utilized in this feature of the invention. More preferably, the non-immunogenic fragment will have a molecular weight in the range of about 0.5 kDa up to about 5 kDa, and especially about 0.5 kDa up to about 2 kDa.

For the purpose of promoting a further understanding and appreciation of the present invention and its advantages, the following specific Examples are provided. It will be understood that these examples are illustrative, and not limiting, of the present invention.

### EXAMPLE 1

### Isolation and Characterization of 65 kDa Protein and Murein Hydrolase Activity

1.1 Materials and Methods

1.1.1 Bacterial strains. S. mutans A32-2 (serotype c), isolated from a caries-active individual, has been previously described (Ray, C. A., et al., Clin. Diagn. Lab. Immunol. 6:400-404 (1999)). S. mutans OMZ175 (serotype f) was obtained from Dr. Suzanne Michalek, University of Alabama at Birmingham. S. mutans NG8 (serotype c) was obtained from Dr. Song Lee, Dalhousie University, Halifax, Nova Scotia. The strains were grown in Todd-Hewitt broth (THB, Difco Laboratories, Detroit, Mich.) plus 1% glucose. Also, ATCC strains S. parasanguis (FW213), S. oralis (10557), S. gordonii (10558), S. sobrinus (27606), and Actinobacillus actinomycetemcomitans (29522) were used in similar testing.

1.1.2 Surface protein preparations. Crude surface protein-enriched fractions of S. mutans A32-2, NG8, UA159 and OMZ175 were prepared as previously described (Perrone, M., et al., Clin. Diagn. Lab. Immunol. 4:291-296 (1997)). S. mutans was grown in 9 L of THB for 18 h at 37° C. in 5% CO₂ and 95% air. Cells were pelleted at 16,274×g at 4° C. for 10 min and washed once gently in Buffer F (10 mM phosphate buffered saline, 1 mM CaCl₂, and 1 mM phenylmethylsulfonyl fluoride (PMSF, pH 7.2)) and stored as a pellet at −20° C. overnight. Frozen cells were thawed and then suspended in Buffer F, and surface proteins were removed by three 1-min cycles at high speed in a Waring blender. After blending, the sample was centrifuged as above to remove intact cells and cell debris. The proteins in the supernatant were isolated by centrifugation (110,000×g, 4° C., 2 h). The pellet containing the surface proteins was resuspended in Buffer F and centrifuged (16,274×g, 4° C., 10 min) to remove cellular debris. The supernatant was divided into aliquots and stored at −80° C. The protein concentration was determined using Quantipro BCA protein assay (Sigma Chemical Co. St. Louis, Mo.). In addition to these surface protein preparations, an isolated sortase enzyme (SrtA, S. mutans NG8) was used.

1.1.3 Peptidoglycan preparation. S. mutans was grown in 3 L of THB for 18 hr. Crude cell walls were prepared according to Hoyle and Beveridge (B. D. Hoyle, et al., Can. Microbiol., 30:204-211, (1984)). Briefly, the cell pellet was washed in 10 mM HEPES, pH 6.8 (Sigma Chemical Co.), and boiled in 4% SDS, 10 mM HEPES for 45 min. The mixture was cooled to RT, centrifuged at 70,000×g for 40 min at 20° C. and washed with 2 M NaCl. The pellet was weighed and resuspended in 2 M NaCl to a concentration of 185 mg/ml and sonicated for 5 min, on ice, at maximum setting.

1.1.4 Electrophoresis and immunoblotting. Unless otherwise indicated, samples were boiled for 10 min in SDS loading buffer and separated by SDS-polyacrylamide gel electrophoresis on 7.5% gels at 150 V for 1 h. Proteins were detected by staining with Coomassie blue. For Western immunoblots, unstained proteins were transferred from SDS-PAGE gels to PVDF nylon membrane at 80 V for 1 h. The membrane was blocked overnight at 4° C. in 5% defatted milk dissolved in washing buffer (0.9% NaCl, 10 mM Tris, pH 7.5). The proteins were detected by incubation with primary antibody (rat anti-surface protein serum (M. Fontana, et al., Indiana University School of Dentistry, Indianapolis, Ind. (1999)), or human monoclonal anti-65 kDa (Epicyte)) followed by horseradish peroxidase-conjugated secondary antibody (anti-rat IgG or anti-human IgG, Sigma Chemical Co.) and color development with 4-chloro-1-napthol (Sigma Chemical Co.). The peptidoglycan zymogram activity gel electrophoresis was performed as previously described (M. Sugai, et al., J. Bacteriol, 172:6494-6498, (1990)). The peptidoglycan preparation (185 mg) was incorporated into the SDS-PAGE gel before pouring. Surface proteins were loaded (without boiling) onto the gel using loading buffer without 2-mercaptoethanol. Following electrophoresis, gels were washed in 250 ml of distilled H₂O for 30 min. The enzyme activity was reconstituted by incubation of gels in 100 mM phosphate buffer, pH 7.0 for 48 hrs, at room temperature. Clear zones of activity were visible in the opaque gel.

1.1.5 Protein sequencing and mass spectrometry. For protein sequencing using a CNBr protocol (Biochemistry Biotechnology Facility, IUPUI), the 65 kDa protein was transferred to PVDF membrane. For analysis using a Micromass MALDI-TOF mass spectrometer (Biochemistry Biotechnology Facility, IUPUI), the Coomassie-stained 65 kDa protein band was excised from the SDS-PAGE gel and digested with trypsin.

1.1.6 Bioinformatics. Mass spectrometric data were compared to data generated from proteins in GenBank using ProFound (http://129.85.19.192/profound_bin/WebProFound.exe). Mass spectrometric data from two individual proteins were compared using Client PAWS™, The Protein Analysis Worksheet. Ex-PASy server (http://www.expasy.ch/) was used to analyze DNA for open reading frames (http://us.expasy.org/tools/dna.html), backtranslation of protein sequence to DNA (http://www.entelechon.com/eng/backtranslation.html), and detection of protein sequences for signal peptide (http://www.cbs.dtu.dk/services/SignalP/) and membrane spanning domains (http://psort.nibb.ac.jp/form.html).

Repeat domains were recognized by visual examination of the protein sequence and used to search. GenBank (http://www.ncbi.nlm.nih.gov/Genbank/GenbankSearch.html) and (http://www.ncbi.nlm.nih.gov/Structure/cdd/cddsrv.cgi?uid=smart00287&version=v1.57) for homologues.

1.1.7 Biofilm Adherence Assay. S. oralis 10557 and S. gordonii 10558 were grown overnight at 37° C. in defined biofilm media (Jenkinson, B. H., Microbiology Micro. 132:1575-1589 (1986)) under anaerobic conditions to OD₆₀₀=0.500. 180 μl bacteria and 10 μg of the cell surface proteins from S. mutans serotype f (OMZ175) or serotype c (A32-2 and NG8) were added to microtiter wells. The plate was incubated overnight, anaerobically, at 37° C. Biofilm formation was detected by staining the cells with 1% crystal violet for 15 minutes followed by 3 washes in distilled water. After air drying, the plates were read on a Thermomax plate reader at 540 λ. Data were analyzed using SigmaStat software.

1.2 Results

1.2.1 DNA and protein analysis of the 65 kDa surface protein. Amino acid sequencing of the 65 kDa Coomassie stained band from the S. mutans A32-2 serotype c surface protein preparation resulted in two 11 aa peptides (MSRQAKAVNIP and MQSPTEFNEDK) which matched (77.3% identity) a predicted 40 kDa cell wall protein from S. mutans OMZ175 serotype f (23) using FASTA to search GenBank (FIG. 1A, dashed underline). The corresponding serotype f DNA sequence was used to search the S. mutans UA159 serotype c genome (1) for the homologous ORF (96% homology) and its predicted translation product (67 kDa) is shown (FIG. 1A). Both proteins have a putative signal peptide and membrane spanning domain, located at amino acid (aa) 1-38 in serotype c and aa 33-70 in serotype f (FIG. 1A, single underline). Three short (46 aa) (FIG. 1A, bold underline) and two long (88 aa) (FIG. 1A, double underline) direct repeats are present in the serotype c ORF, while only one copy of the short repeat is present in the serotype f ORF (FIG. 1A, bold underline).

Both ORF's (for the 67 kDa predicted gene product from UA159 serotype c and the 40 kDa predicted gene product from OMZ175 serotype f) are located immediately upstream from the coding region for cell surface protein antigen I/II (termed sr in serotype f) (23) (FIG. 1B).

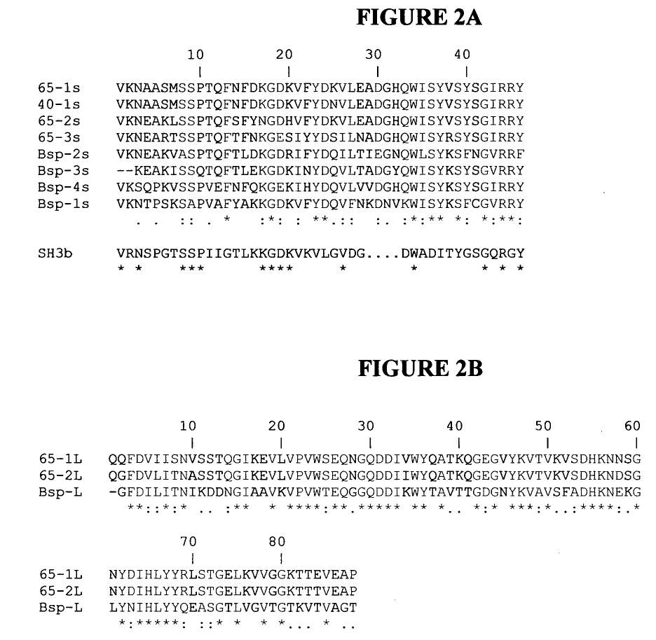

FIG. 2 illustrates the homology between the repeat regions of S. mutans c, S. mutans f and those of a putative murein hydrolase, Bsp, from S. agalactiae (D. J. Reinscheid, et at., Microbiol., 148:3245-3254, (2002)). The short repeat region (46 aa) is also compared to the consensus sequence for a bacterial motif, SH3b (32), named for weak homology to eukaryotic SH3 domains, and is associated with amidases (V. Lazarevic, et al., J. Gen Microbiol., 138:1949-1961, (1992)).

Comparison of the corresponding nucleotide sequence of S. mutans UA159 serotype c (using GenBank BLAST search of microbial genomes) with the sequence encoding the putative cell wall protein from S. mutans OMZ175 serotype f (FIG. 3) revealed the presence of a stop codon in serotype f at nt 1103-1105 which results in a protein of the predicted mass of 40 kDa, while in UA159 serotype c, the same open reading frame, designated SMU.609, (1) extends to a predicted protein size of 67 kDa. A single base pair deletion at nt 1016 in OMZ175 ORF (bold underline) resulted in a frameshift mutation in serotype f which produced the early stop codon. The homology between the two genomes extends beyond the coding region, but due to the frameshift mutation, it is not apparent in the protein sequence.

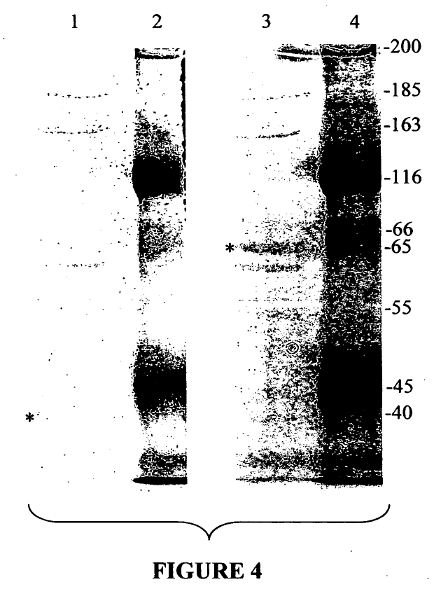

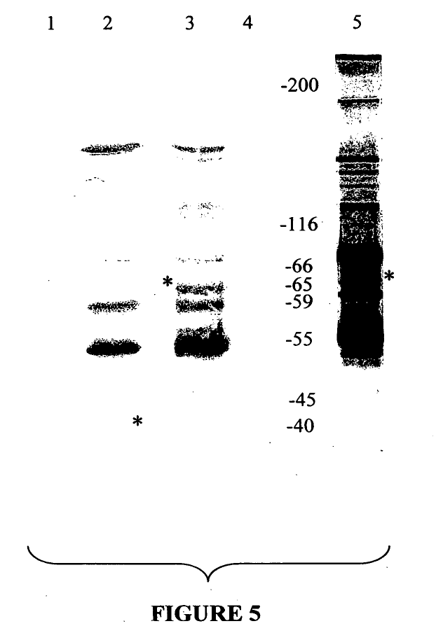

1.2.2 Comparative analysis of surface proteins. Comparison of Coomassie stained surface protein preparations from the two serotypes (FIG. 4) revealed the presence of a protein at 40 kDa in OMZ175 serotype fwhich is absent in A32-2 serotype c and that serotype flacks the 65 kDa protein which is present in serotype c. Probing Western blots of these surface protein preparations with antibody to S. mutans A32-2 crude surface proteins (FIG. 5) confirms that the immunoreactive 65 kDa protein is absent from serotype f but the 40 kDa protein in serotype f (absent from serotype c) is recognized by this antibody.

1.2.3 Mass spectrometric analysis of the 65 kDa protein. Mass spectrometric analysis of the predicted protein (Client PAWS, The Protein Analysis Worksheet) encoded by the UA159 serotype c ORF matches the actual mass spectrometric data generated by trypsin digestion of the gel-excised 65 kDa S. mutans (A32-2)-produced protein, while the virtual mass spectrometric analysis of the predicted protein from the homologous ORF found in OMZ175 serotype fmatched the mass spectrometric data from the gel-excised 65 kDa protein from A32-2 serotype c most closely in the homologous regions of the two proteins (not shown).

1.2.4 Functional analysis of the 65 kDa protein. Zymogram activity gels reveal several bands of autolytic murein hydrolase activity in the non-denatured surface protein preparation from S. mutans A32-2 serotype c (FIG. 6A, lane 1), one with an apparent molecular weight of 65 kDa. No activity was associated with the surface preparations from S. mutans serotype f (FIG. 6A, lane 2). Identical results were obtained using peptidoglycan from serotype c (FIG. 6A, lanes 3-7), with hydrolytic activity associated with serotype c surface protein preparations (A32-2, lane 3; NG8, lane 4; UA159, lane 7) and no hydrolytic activity associated with the serotype f surface protein preparation (OMA175, lane 6) or with the SrtA enzyme (lane 5). In additional zymogram activity gel testing, the serotype c surface protein preparations also exhibited hydrolytic activity against Streptococcus sobrinus (FIG. 6A, lane 8), Streptococcus gordonii (FIG. 6A, lane 9), Streptococcus oralis (FIG. 6A, lane 10), and Actinobacillus actinomycetemcomitans (FIG. 6B). The results of the biofilm adherence assays are shown in FIG. 6C. The results reveal that S. mutans serotype c surface protein preparations containing the 65 kDa protein inhibited the adherence of S. gordonii and S. oralis to microtiter wells, whereas the surface protein preparation from S. mutans OMA175 (serotype f) did not.

1.3 Discussion

Evidence supports that the 65 kDa protein in S. mutans A32-2 (seroptype c) surface protein preparations is the full length protein homologous to a previously described 40 kDa putative cell wall protein, (J. A. Ogeir, et al. Infect. Immun., 59:1620-1626 (1991)) present in S. mutans serotype f surface protein preparations. Regions of homology have been determined by protein sequencing, mass spectrometric similarities and differences between the isolates have been noted, the DNA sequence comparison reveals the exact differences between representatives of the two serotypes at the coding level, and immunoblot analysis of surface preparations confirming the absence of the 65 kDa protein in serotype fand the corresponding presence of the 40 kDa protein.

As to the S. mutans serotype c ORF predicted to encode the 65 kDa protein, analysis has revealed the presence of three short (46 aa) and two long (88 aa) direct repeat regions, an N-terminal signal sequence, and a membrane spanning domain. This is consistent with the fact that cell surface proteins of many Gram-positive bacteria contain repetitive domains which may serve as virulence determinants (H. Wu, et al. Molecular Microbiology, 34:1070-1081 (1999)), or have a role in protein-protein interactions. In addition, a putative murein hydrolase, Bsp, (60 kDa) from S. agalactiae (D. J. Reinsheid, et al. Microbiol., 148:3245-3254 (2002)) has four regions homologous to the short repeat of S. mutans 65 kDa protein. Bsp also has one region homologous to the long repeat which is found twice in the S. mutans 65 kDa protein. The repeats in the S. mutans 65 kDa protein are arranged as two short, two long, one short. In Bsp, the arrangement of the homologous repeat regions is two short, one long, two short. D. J. Reinscheid, et. al. Microbiol. 148:3245-3254 (2002) were unable to show autolytic activity associated with a Bsp-fusion protein or with Bsp in culture supernatants using a zymogram technique. However, it is unclear whether they tried using non-denaturing conditions as described in the Examples below.

The short repeat regions in the 65 kDa protein have homology to a common bacterial motif, (J. C. Whisstock, et al., Annu. Rev. Microbiol., 50:513-552 (1996)) referred to as SH3b domains because of its weak homology to eukaryotic SH3 domains. The initial SH3b domain was identified in the iap gene of Listeria grayi, which is thought to possess a murein hydrolase activity, (M. D. Wuenscher, et al., J. Bacteriol, 175:3491-3501 (1993)). Other examples of proteins with homologous SH3b domains include S. pneumoniae choline binding protein D (two copies) (J. Hoskins, et al., J. Bacteriol, 183:5709-17 (2001)); a putative choline binding protein from S. pyogenes (one copy) (J. J. Ferretti, et al., Proc Natl Acad Sci USA, 98(8):4658-63(2001)); (and the glycyl-glycine endopeptidase gene from Staphylococcus aureus (one copy) (P. A. Recsei, et al., Proc Natl Acad Sci USA, 84:1127-31 (1987)). None of these have homology to the 88 aa long repeat found in the 65 kDa protein from S. mutans serotype c.

The gram-positive peptidoglycan layer presents a barrier to export of large protein subunits, such as those present in our crude surface protein preparation (A. J. Dijkstra, et al., J. Bacteriol 178:5555-5562 (1996)). A murein hydrolase associated with these proteins may be important in transport to the cell surface. Data suggest the 65 kDa protein present in the S. mutans serotype c surface protein preparation is an autolytic murein hydrolase. Lytic activity is associated with multiple bands from the serotype c crude surface protein preparation. The reducing agent, 2-mercaptoethanol, was omitted from the zymogram gel loading buffer (M. T. Sugai, et al., J. Bacteriol 172:6494-6498 (1990)) because it has been reported to interfere with reconstitution of autolytic enzymes. Western immunoblot data obtained under these electrophoretic conditions indicates the 65 kDa protein was incompletely dissociated from other surface components and may be responsible for all the activity detected. The data do not rule out the possibility that such association with another surface protein may, in fact, be required for the maximum activity apparent at the higher molecular weights. Alternatively, multiple murein hydrolase enzymes may have been purified with the crude surface proteins, and one such enzyme is responsible for some of the activity seen. GBP-B (55 kDa), present in our surface protein preparation, has been reported to have homology to PcsB from S. agalactiae, and to P45, a protein from Listeria monocytogenes that was identified as a peptidoglycan hydrolase (R. O. Mattos-Graner, Infect. Immun., 69:6931-6941 (2001)). However, no autolytic activity has been reported for GBP-B or PscB. GBP-B is also present, at the expected molecular weight, in the surface proteins isolated from S. mutans serotype f (55 kDa band seen in FIG. 5). No hydrolase activity was associated with the surface proteins from S. mutans serotype f Therefore, it is unlikely that GBP-B is responsible for the activity seen with serotype c surface proteins.

Recently, (D. Ajdic, et. al. Proc Natl Acad Sci USA, 22:14434-9 (2002)), analyzed the S. mutans UA159 genome for ORF's and identified 6 with homology to known autolysins. These ORF's were designated according to their position on the genome as SMU.76, SMU.574, SMU.704, SMU.707, SMU.1700, SMU.2174. Each of these ORF's is predicted to encode a protein smaller than 65 kDa. Their analysis did not identify SMU.609 (the ORF we have identified as encoding the 65 kDa protein) as a potential murein hydrolase, but identified it as a 40 kDa cell wall precursor protein, presumably due to its homology to the ORF described as such from serotype f (J. A. Ogier, et al., Infect. Immun., 59:1620-1626 (1991)). Based on our analysis, SMU.609 should be listed as a potential murein hydrolase.

S. mutans serotype c is isolated from the oral cavity more frequently than is serotype f. (S. Hamada, et al., J. Clin. Microbiol., 11:314-318 (1980)). Thus, serotype c isolates seem to possess a colonization advantage over serotype fisolates. Many factors could contribute to this difference including ability to utilize nutrients, ability to adhere efficiently to the pellicle-coated tooth surface, ability to form multicellular complexes, and ability to evade host defenses. Differences between cell surface proteins—such as seen in this report between the 65 kDa and 40 kDa homologs—may contribute to differences in prevalence or virulence of the two serotypes.

Due to the fact that the 65 kDa protein is recovered with other surface proteins having well-established roles in adhesion and that the gene encoding the 65 kDa protein is located immediately upstream from the ORF SMU.610 (1) encoding a known adhesion molecule, Cell Surface Antigen I/II, we hypothesize that the 65 kDa protein has a role in adhesion. (C. Heilmann, et al., Mol. Micro. 24:1013-1024 (1997)) reported that an autolysin from Staphylococcus epidermidis, AtlE, also functions in adherence of the bacterium to polystrene and to plasma protein—coated polymer surfaces. Likewise, a Listeria monocytogenes autolysin, Ami, has been shown to be an adhesin (E. Milohanic, et al., Mon. Micro, 39:1212-1224 (2001)), both via interactions mediated by repeat domains.

Many prokaryotic proteins that are expected to insert into or cross membranes are initially made in a precursor form with an N-teminal signal sequence (W. Joklik, et al., Zinsser Microbiology, p 47, 19^{th} Edition). The signal sequence of these protein precursors is removed by signal peptidase during or shortly after translocation across the cytoplasmic membrane. The presence of a signal sequence in the translation product of the serotype c ORF supports the idea that this sequence encodes a cell surface protein, consistent with the purification properties of the 65 kDa protein.

The 40 kDa truncated version of this protein from serotype f has the same signal sequence and membrane spanning domain, however they are located 33 aa away from the predicted N-terminus. A 40 kDa protein is present in the crude surface protein preparation from serotype fwhich is consistent with the fact that it is predicted to be found on the surface of the bacterium.

These data reveal a difference in the molecular weight and motif composition of a surface protein produced by two serotypes of S. mutans (c and f) due to a frameshift mutation. Additionally, these data show a difference in function between the serotypes c and f surface proteins. A murein hydrolase active against both serotype c and f peptidoglycan is present in the crude surface protein preparation from S. mutans serotype c (A32-2 and NG8). No hydrolase activity was detected using the surface protein preparation from S. mutans OMZ175 serotype fagainst peptidoglycan from either source.

### EXAMPLE 2

### Cloning of Open Reading Frame Encoding the 65 kDa Protein

Standard PCR cloning techniques are used to clone the gene for the 65 kDa protein from S. mutans UA159 and A32-2 strains. Primers are generated based on the DNA sequence of UA159 available in GenBank. An E. coli expression vector, such as pET100/D-TOPO (Invitrogen, Carlsbad, Calif.), is used according to manufacturer's directions. Should expression of S. mutans murein hydrolase be lethal in E. coli, the gene is cloned in a commercially available yeast or insect expression systems, using PCR cloning into vectors such as Invitrogen's pPICZ or pIB/V5-His respectively.

### EXAMPLE 3

### Preliminary Purification and Characterization of 65 kDa Protein Preparation

3.1 Materials and Methods

3.1.1 Bacteria. A S. mutans isolate from the saliva of a 7 yr old caries active child (defined as having ≧5 unrestored surfaces and designated strain A32-2) was used in all experiments and was maintained in 5% CO₂ and 95% air at 37° C. overnight in Todd Hewitt broth (Difco Laboratories, Detroit, Mich.) and passed a minimum number of times. This strain has previously been described to be heavily fimbriated.

3.1.2 Fimbrial preparation. A modification (M. Fontana et al, Clin. Diaa. Lab. Immun. 2: 719-725 (1995); M. Perrone et al., Clin. Diag. Lab. Immun. 4: 291296 (1996)) of the technique of McBride and colleagues (E. J. Morris et al., J. Bacteriol. 169: 164-171 (1987)) for isolating fimbriae from S. sanguis whole cells, was used for the removal of S. mutans fimbriae. The procedure utilized alternating high and low speed centrifugations. S. mutans was grown in nine liters of Todd Hewitt broth for 18 h at 37° C. in 5% CO₂ and 95% air. Cells were pelleted and washed once gently at 16,274×g at 4° C. for 10 min in fimbriae buffer (10 mM phosphate-buffered saline, 1 mM CaCl₂ and 1 mM phenylmethylsulfonyl fluoride [PMSF]; pH 7.2) and frozen as a pellet at −20° C. overnight. PMSF was added to inhibit endogenous proteolytic digestion of fimbrial proteins and CaCl₂ was used to reduce fimbrial aggregation. Frozen cells were thawed, suspended in fimbriae buffer and fimbriae were removed by a Waring blender using two one-min cycles at high speed. Following blending, the sample was centrifuged (16,274×g, 4° C., 10 min) to remove intact cells and cell debris and the fimbriae preparation in the supernatant was isolated by ultracentrifugation (110,000×g, 4° C., 2 h). The pellet containing the fimbriae preparation was resuspended in fimbrial buffer and centrifuged (16,274×, Rockford, Ill.) and stored at −80° C. The protein concentration was determined using the Bio-Rad micro-protein assay (Bio-Rad Laboratories, Hercules, Calif.).

3.1.3 Preparation of salivary components and the purified fimbrial protein. Saliva was collected from seven healthy individuals [neither caries free (no decayed, missing or filled surfaces) nor caries active (≧5 unrestored surfaces)] and stored at −20° C. Prior to use, the salivas were clarified by centrifugation (2,800×g, 4° C., 10 min) and protein concentrations determined. Saliva samples were diluted to 500 μg of protein/ml in physiological saline for SDS-PAGE or in 0.1 M carbonate/bicarbonate buffer (pH 9.6) for ELISA. In order to separate Salivary protein fractions, preparative gel electrophoresis (Prep cell model 491; Bio-Rad) was utilized. The resolving and stacking gels were composed of 10% and 3% acrylamide (National Diagnostics, Atlanta, Ga.), respectively. A clarified whole saliva sample (2 ml) was added to an equal volume of SDS-PAGE sample buffer, boiled for 7 min and placed on a 6 cm column and subjected to 12 W of continuous power. The protein of interest eluted after approximately 8 h of electrophoresis and was previously determined by immunoblots of whole saliva to have a molecular weight of approximately 52 kDa. The proteins were collected and analyzed for molecular weight and purity by gel electrophoresis after staining with coomassie brilliant blue. The fractions of interest were pooled and passed through an affinity column that removes SDS (Extracti-Gel, Pierce, Rockford, Ill.) and stored at −80° C. Purification of the immunodominant 65 kDa fimbrial protein identified earlier (M. Fontana et al, Clin. Diag. Lab. Immun. 2: 719-725 (1995); M. Perrone et al., Clin. Diag. Lab. Immun. 4: 291296 (1996)) was also accomplished by preparative gel electrophoresis using an identical method. Rat antisera to the enriched A32-2 fimbrial preparation and to the 65 kDa fimbrial protein were obtained from eight animals each immunized with 5 Rg protein/ml incorporated into the RIBI adjuvant system (RIBI ImmunoChem Research, Inc., Hamilton, Mont.). Preparations were injected with 0.2 ml s. c. in each of two sites and 0.1 ml i. p. twice 21 days apart and blood collected 7 days after the last injection. The blood was allowed to clot and serum was obtained and frozen at 20° C. until used.

3.1.4. ELISA assay for binding of fimbriae and fimbrial protein to whole saliva and salivary components. Whole saliva (undiluted and 1:2 and 1:10 dilutions), purified 52 kDa salivary protein (65.0 μg/mi) and human salivary a-amylase (10.0 μg/ml; Type IX A, Sigma Chemical Co., St. Louis, Mo.) were assayed to determine the ability of fimbriae and the fimbrial protein to bind to salivary components. Polystyrene 96 well microtitre plates (Flow Laboratories, Inc., McLean, Va.) were coated (100 μl) with the salivary components or whole saliva (diluted in 0.1 M carbonate/bicarbonate buffer; pH 9.6) and incubated for 3 h at 37° C. or overnight at 4° C. The unbound salivary components were removed by washing three times with normal saline containing 0.05% Tween 20 (Tween-saline). A solution of 1% bovine serum albumin (BSA; Sigma) in carbonate/bicarbonate buffer was added (200 μI) to block any unbound sites and incubated for 1 h at 25° C. Following washing, 100 μl of the A32-2 fimbrial preparation (33.0 μg/ml in saline), purified 65 kDa fimbrial protein (1 μg/m or Tween-saline (no fimbriae control) was added and incubated for 3 h at 37° C. and washed 3 times. Rat antibody to the A32-2 fimbriae preparation or rat anti-65 kDa fimbrial protein (both diluted 1:4000 in Tween-saline) was added (100 μl) and incubated for 3 h at 37° C. After washing, goat antibody to rat IgG (Fc specific) conjugated to horseradish peroxidase (Sigma) was added (100μ; 1:8000 dilution) and incubated for 3 h at 37° C. After a final wash step, the substrate (10 mg of orthophenylenediamine dihydrochloride and 14 μl of 30% H₂O₂ in 20 ml of 0.5 M citrate buffer [pH 5.0]) was added (100, μl) and developed for 30 min and read at 490 nm using a microplate spectrophotometer (Molecular Devices Corp., Menlo Park, Calif.). In addition, a modification of the ELISA assay described above was used to determine the efficacy of the purified 52 kDa salivary protein in inhibiting the binding of S. mutans A32-2 fimbriae to a 1:10 dilution of whole saliva. Mixtures of the S. mutans fimbriae preparation (33.0 μg/ml) and serially diluted 52 kDa salivary protein (0.5-65.0 μg/ml) were incubated for 30 min at 37° C. and used in place of the untreated fimbriae preparation. Controls included whole saliva and BSA.

3.1.5. Immunoblot analysis for binding of fimbriae to salivary components and amylase detection. In order to determine which components in whole saliva bound S. mutans fimbriae, reducing sodium dodecyl sulfate-polyacrylamide gel electrophoresis (SDS-PAGE) was used (U. K. Laemmli, Nature 227: 680-685 (1970)). The resolving and stacking gels were composed of 10% and 3% acrylamide, respectively. Saliva samples (50 μl samples in saline) were boiled for 7 min and electrophoresed using a mini-gel electrophoresis apparatus (Mini-Protean II; Bio-Rad) for 60 min at a voltage of 150V. After electrophoresis, separated proteins on the gel were transferred to nitrocellulose paper (BioRad) overnight at 4° C. at a constant voltage of 30V in a mini-transblot electrophoretic transfer cell (31; Bio-Rad). The nitrocellulose paper was blocked in a solution of 1% defatted milk (Carnation instant milk, Carnation Company, Los Angeles, Calif.) diluted in washing buffer (0.9% NaCl containing 0.5% Tween-20; WBT) for 2 h at 25° C. The nitrocellulose paper was washed with WBT 3 times for 10 min each, the S. mutans fimbrial preparation (33 μg/ml; 2 ml) in WBT was added and incubated for 1 h at 25° C. The membrane was washed to remove unbound protein and incubated with rat antibody to A32-2 fimbriae (diluted 1:500 in WBT) for 1 h at room temperature. Goat antibody to rat IgG (Fc specific)-alkaline phosphatase conjugate (1:1000 in WBT; Sigma; 100 μl) was added and incubated for 1 h. Binding of the antibody was detected by addition of alkaline phosphatase substrate (p-nitroblue tetrazolium chloride and 5bromo-4-chloro-3-indoyl phosphate; Bio-Rad) dissolved in 100 mM Tris HCl (pH 9.5). In order to determine whether the 52 kDa salivary protein was amylase, the isolated salivary protein (65.0 μg/ml), commercial purified amylase (10.0 μg/ml) and undiluted whole saliva were electrophoresed in SDS-PAGE, transferred to nitrocellulose and probed with rabbit anti-human a amylase (Sigma) followed by alkaline phosphatase-labeled goat antirabbit IgG (Sigma) and substrate similar to the method described above.

3.1.6. Statistical analysis. The data were reduced by computing the means and standards errors of the mean of the absorbances of triplicate determinations per sample. The data were analyzed by Student's t test and significant differences were defined as p≦0.05.

3.2 Results

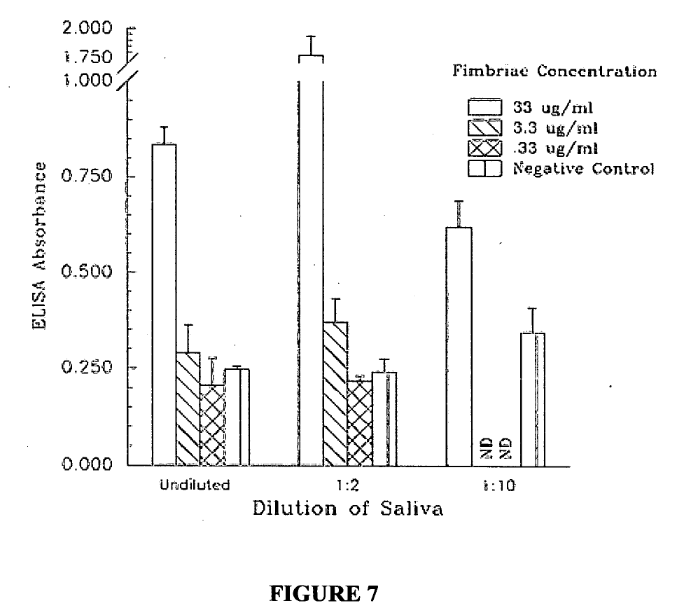

3.2.1. Fimbriae binding assays. ELISA and immunoblotting were used to establish binding of the S. mutans fimbriae preparation to saliva-coated surfaces. An ELISA was performed to determine if a S. mutans fimbriae preparation from strain A32-2 bound to human whole saliva. Fimbriae from S. mutans A32-2, a strain isolated from a caries active subject, demonstrated significant binding as compared with the corresponding Tween-saline control (no fimbriae; FIG. 7). The binding of fimbrial components to saliva was reduced when either the saliva or fimbriae was diluted. These data provided the first indication that S. mutans fimbriae had binding activity with saliva-coated surfaces. BSA-coated wells did not bind fimbriae (data not shown).

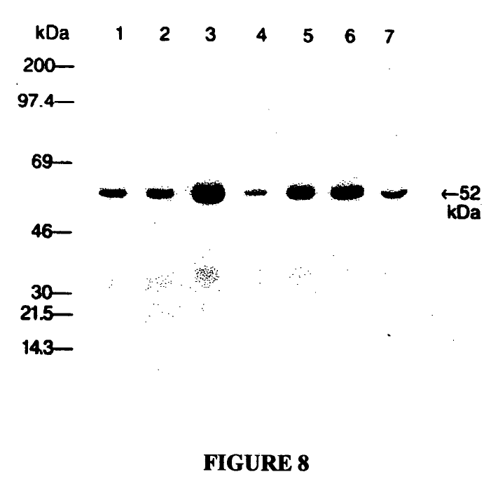

3.2.2. Immunoblot analysis of human whole saliva probed with S. mutans A32-2 fimbriae. The binding activity of the S. mutans A32-2 fimbriae preparation to separated salivary proteins was analyzed by immunoblotting. Human whole saliva samples were collected from seven healthy subjects. Each saliva sample was electrophoresed, transferred to nitrocellulose paper and probed with the S. mutans fimbriae preparation. Fimbriae from the A32-2 strain bound strongly to a 52 kDa salivary protein in all 7 saliva samples (FIG. 8). Controls with no fimbriae did not reveal any bands.

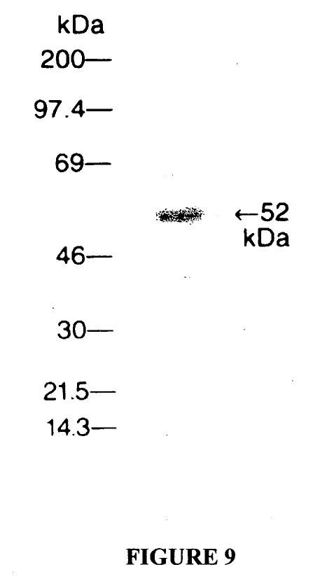

3.2.3. Isolation of a 52 kDa salivary protein with S. mutans fimbriae binding activity. In order to better understand the interaction between the 52 kDa salivary protein and S. mutans fimbriae, the salivary protein was isolated by preparative gel electrophoresis. Following elution, the fractions were analyzed by gel electrophoresis and fractions were identified that contained only one band (FIG. 9).

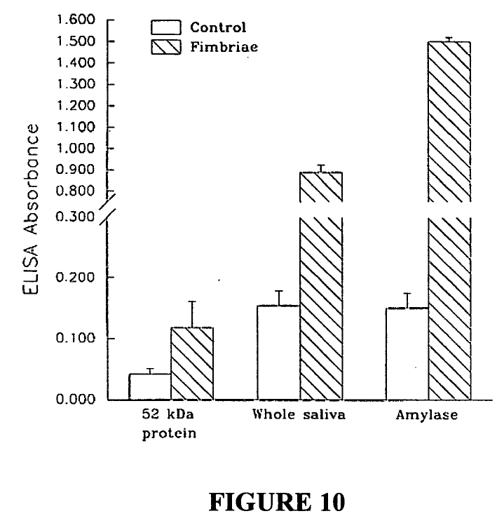

3.2.4. ELISA assay for binding of S. mutans fimbriae and purified 65 kDa fimbrial protein to isolated salivary protein, amylase and whole saliva. In order to ascertain that both the salivary protein and amylase have fimbrial-binding characteristics, an ELISA was employed to measure binding. Amylase was chosen because the molecular weight was similar to 52 kDa and several oral streptococci have demonstrated the ability to bind to amylase. In this assay, amylase (10.0 μg/ml) had significantly greater fimbrial-binding activity than the no fimbriae Tween-saline control (FIG. 10). Amylase also had a significantly greater absorbance compared to diluted whole saliva (0.5 μg/ml). The isolated salivary protein (65.0 μg/ml) had a lower absorbance than either amylase or whole saliva, but the absorbance was significantly higher than the no fimbriae control. Purified 65 kDa fimbrial protein bound similarly to amylase (O. D. 0.250+0.026) as to a 1:2 dilution of saliva (O. D. 0.260+0.030), but not to a Tween-saline negative control (0.070+0.012).

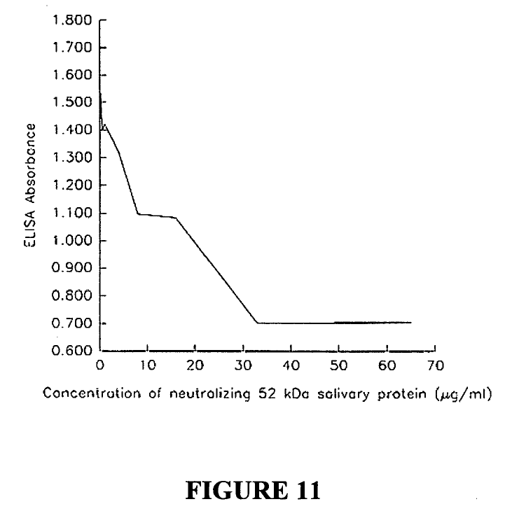

3.2.5. Inhibition of binding of S. mutans fimbriae to whole saliva-coated surfaces. In binding assays, an important feature is the ability to inhibit the interaction. The ability to inhibit binding suggests that the interaction is specific. In this system, the purified salivary protein was incubated with the fimbriae preparation from S. mutans A32-2. Following incubation with the salivary protein, the mixture was added to whole saliva. The data indicated an inverse relationship between the concentration of the salivary protein and the extent of binding of the S. mutans fimbriae preparation to whole saliva (FIG. 11). Whole saliva and BSA controls indicated complete and no inhibition, respectively.

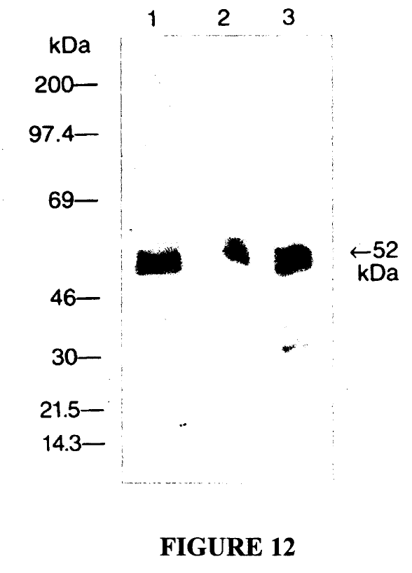

3.2.6. Immunoblot analysis of the purified salivary protein probed with anti-human α-amylase antibody. The purified salivary protein, human amylase and whole saliva were assayed for reactivity with rabbit antibody to human a-amylase. The results indicated that all three salivary preparations contained components that were recognized by the antiamylase antibody (FIG. 12).

### EXAMPLE 4

### Analysis of 65 kDa Protein for Inhibition of Plaque and/or Dental Caries

Polypeptides (including fragments) are screened for their ability to inhibit plaque and/or dental caries formation as follows.

4.1. Creation of 65 kDa protein knockout mutant. Using the available DNA sequence of S. mutans, a PCR based insertion-deletion strategy (Y. Li, et. al., J. Bacteriol. 184:2699-2708 (2002)) is used to create a specific 65 kDa protein knockout mutant. The DNA region immediately upstream from the ORF encoding the 65 kDa protein is amplified using specific primers, with a restriction enzyme site added to the 3′ primer. Using this restriction site, the PCR product is ligated to a compatible restriction site on the 5′ end of a PCR amplified erythromycin (ERM) resistance marker, such as that found in the ERM-resistant plasmid pTV1-OK (Q. Chen, et. al., Infect. Immun. 70:6534-6540 (2002)). Likewise, the region immediately downstream from the ORF encoding the 65 kDa protein is PCR amplified using specific primers with a different restriction site added to the 5′ primer. This restriction site is used to ligate the downstream PCR product to the compatible site on the 3′ end of the ERM cassette. The final construct is transformed into competent S. mutans A32-2 (D. Perry and H. K. Kuramitsu, Infect. Immun. 32:1295-1297 (1981)). Following double-crossover homologous recombination, the 65 kDa ORF is replaced by the ERM cassette.

4.2. Analysis of colonization and cariogenicity of knockout mutants using an in vitro model. For each study, four groups of 12 human teeth specimens per group are treated for a 4-day test period in an in vitro microbial artificial-mouth caries model. The groups will differ from each other in the presence (A1, A2, and A3) or absence (B1: negative control) of S. mutans (serotype c), and in the concentration of 65 kDa (10-500 μg/ml) used. Enamel specimens (3 mm in diameter) are drilled from extracted, sound, human, lower permanent incisors which had been obtained from oral surgeons and sterilized by soaking in 3% buffered (neutral) formalin since the time of extraction. Each specimen is mounted on a polyacrylic rod using methyl methacrylate resin. The specimens are ground using 600-grade silicon carbide paper to remove approximately 50 lum of the surface and then polished to a high luster with Gamma Alumina (0.05 μm) using standard methods. The specimens are then randomly assigned to test groups, each group initially composed of 14 specimens. All specimens are sterilized with ethylene oxide gas. Two specimens from each group are randomly chosen before treatment and examined to obtain baseline confocal microscopy data. The 12 specimens that remained in each group are secured in caries-forming vessels by gluing the ends of their plexiglas rods to a round plexiglas base that fit in the bottom of the vessels. Trypticase soy broth without dextrose (Difco Laboratories; Detroit, Mich.) (TSBS) is used as the bacterial nutrient broth. For each caries-forming vessel there is one 1 L bottle of TSBS that dispensed the media (0.7 ml/min) at three different times each day, for 30 min each, for a total of 63 ml/day by means of a peristaltic pump (Wiz peristaltic pump; ISCO, Inc., Lincoln, Neb). A mineral wash (MW) solution (pH 7.0) is used to mimic the action of saliva. One liter of MW contained: potassium chloride (624.6 mg); sodium chloride (866.6 mg); dipotassium hydrogen phosphate (33.8 mg); magnesium chloride (59.6 mg); and calcium chloride dihydrate (166.6 mg). Twenty-liter polypropylene bottles (Fisher Scientific; Pittsburgh, Pa.) are used to store the MW. There is one bottle for each of two groups. Each bottle dispenses approximately 882 ml of MW/day (0.7 ml/min) to each caries-forming vessel, intermittently over a period of 21 h, during the periods with no TSBS flow, by a peristaltic pump. Three times a day, prior to each TSBS cycle, 1 ml of the appropriate 65 kDa protein solution is administered to each group by injection, followed by flushing with 5 ml of sterile saline (8.78 g NaCl/L deionized water). The antibody solutions are allowed to mix with fluid (MW) in the caries-vessels by stirring for 30 min before cycling is resumed. All of the media and model components are autoclaved at 121° C. for 20 min prior to initiation of each experiment. Each group of 12 specimens is placed in a caries-forming vessel (125-ml Pyrex brand slow speed stirring vessel; Fisher). All caries-forming vessels are placed on an electric stirrer inside an incubator at 37° C. under aerobic conditions. Each caries vessel has three inlets, one for TSBS, one for NW, and one for injection of the antibody; and one outlet for drainage tubing. The drainage tubing ended flush in a drainage container, which is also placed inside the incubator. Drainage of fluid from each caries vessel is maintained at 0.7 ml/min by a peristaltic pump (Wiz, ISO). Each specimen in groups A1, A2, and A3 is inoculated by micropipette with 20 μl of washed, overnight (16 h), stationary-phase cells of S. mutans A32-2 (serotype c), resuspended in TSBS to an optical density of 0.5 OD at 540 nm. Group B1, the negative control, is inoculated with TSBS only. Prior to inoculation, filter (0.2 μm)-sterilized 65 kDa protein is placed on the surface of the uncoated or amylase-coated (200 μg/ml) enamel specimens (20 μl) and incubated for 1 h at 37° C. in order for the 65 kDa protein to prevent biofilm plaque formation. Sterile saline (8.78 g NaCl/L of deionized water) is used instead of the 65 kDa protein in group A1. Following inoculation with 20 μl of bacteria the specimens are incubated for two hours at 37° C. to allow the bacteria to implant on the teeth. Each group of specimens is then placed in a separate caries forming-vessel and attached to the MW, TSBS/TSB and drainage container bottles. Alternatively, the 65 kDa protein is added to the caries-forming vessel 1 to 2 days after the initiation of the S. mutans biofilm plaque layer in order to disrupt the plaque layer. The following parameters are measured in the supply and drainage containers fluid at the beginning and at the end of the 4-day test periods to monitor the absence of contamination and the viability of the inoculum: 1) pH; 2) S. mutans viability (by plating on mitis salivarius agar, Difco; supplemented with 20% sucrose and 200 IU/L bacitracin); and 3) bacterial contamination (by plating on trypticase soy agar, Difco). Plates are incubated at 37° C. in 5% CO₂-95% air for 3 days. In order to quantitate the bacteria adhered to the teeth, at the end of the test periods teeth from each group are randomly selected and placed individually in 5 ml of sterile saline. Each specimen is then vortexed (20 sec) and sonicated (20 sec) until all visible dental plaque is displaced from the surface of the tooth. All samples are then plated on mitis salivarius and trypticase soy agar. Following termination of each study, caries activity is assessed using standard procedures (Fontana M., A. J. Dunipace, R. L. Gregory, T. W. Noblitt, Y. Li, K. K. Park, G. K. Stookey. 1996. An in-vitro microbial model for studying secondary caries formation. Caries Res. 30: 112-118).

4.3. Analysis of inhibition in a rat caries model. The ability of the 65 kDa protein and fragments to inhibit colonization and caries lesion formation by S. mutans is assessed using a standard rat caries model. Briefly, thirty conventional rats (Harland Sprague Dawley) are used per group. Each polypeptide is assigned to a separate group. At 15 days of age, each rat is challenged with wild type A32-2 on four days (15 and 18-20 days of age). One rat group serves as a positive control (no administration of polypeptide), and another control group is not infected. Other groups are administered the polypeptide in their drinking water at levels varying from 10-500 μg/ml. All animals are fed a standard rat chow diet. Saliva and serum samples are collected at sacrifice (12 weeks after infection; 104 days of age) to determine salivary IgA and serum IgG antibody levels to S. mutans A32-2 whole cells, and polypeptide using general methods as described earlier (M. Fontana et al., Clin. Diagn. Lab. Immunol., 2: 719-725 (1995)). At termination, mandibles are assessed for adhered S. mutans levels, and then scored for caries. The animals are euthanized during blood collection by intracardiac puncture. The hemijaws (maxilla, mandible) are surgically removed, and the lower right jaw aseptically freed of all soft tissue using a sterile scalpel. This hemijaw (lower right) is used for bacterial counts, and later returned for pressure cooking (10 PSI for 12 min) with the other three hemijaws (lower left, upper right and upper left) for caries scoring. Pressure cooking is done to facilitate soft tissue removal prior to caries scoring. One hemijaw quadrant (right-mandibular) is placed in a tube containing 3 ml of sterile saline (8.77 g NaCl/L of dH20), and plaque disrupted from the molar surfaces by vortexing for 20 sec, followed by sonication for 20 sec at a setting of 20 (50 Sonic Dismembrator, Fisher), and finally vortexing again for 20 sec. The number of S. mutans cells in the plaque samples is determined by culturing known dilutions (undiluted and 1:10; double plated) of samples, using a Spiral Plater (Spiral Systems) on mitis salivariusrifampicin and fusidic plates, and incubated at 37° C. All hemijaws (four quadrants) are stained overnight with a murexide (Sigma) solution (0.3 g of murexide; 300 ml of dH₂O and 700 ml of ethanol) for caries scoring. The jaws are rinsed and allowed to dry. The jaws are examined for smooth surface caries, sectioned, and then microscopically examined for sulcal and interproximal caries using the Keyes method (P. H. Keyes, J. Dent. Res., 37: 1088-1099 (1958)).

### EXAMPLE 5

### Preparation of Amylase Binding Fragments

A purified preparation of the identified 65 kDa S. mutans protein is subjected to tryptic digestion with 10 μg/ml for 30 minutes at 37° C. The sample is separated by SDS PAGE using a 10-20% gradient gel. The separated peptides are transferred from the gel to immunoblotting paper and probed with 10 μg/ml human salivary alpha-amylase (Sigma Chemical Co.) for 1 hour at room temperature. After washing the blot, rabbit anti-human amylase antibody is added and incubated for 1 hour, washed and alkaline phosphatase-labeled goat anti-rabbit IgG antibody added and incubated for 1 hour. After washing, NBT/BCIP substrate is added for color development. The peptide band or bands that bind amylase will have a color change and their molecular weight is determined. After establishing the molecular weight of the amylase binding peptide, the peptide is purified from a tryptic digest by preparative electrophoresis and can be used in functionally blocking S. mutans adherence to the salivary pellicle.

While various preferred embodiments of the invention have been described in detail above, the same is to be considered illustrative in nature. All modifications and additions as would occur to one of ordinary skill in the field to which this invention pertains are contemplated as being a part of this invention and are desired to be protected. In addition, all publications cited herein are indicative of the level of ordinary skill in the art, and are hereby incorporated by reference in their entirety as if each had been individually incorporated by reference where cited and fully set forth.

1. A method for controlling dental plaque in the oral cavity of a host, comprising administering to the oral cavity of the host an effective amount of a polypeptide having S. mutans murein hydrolase activity against one or more bacterial species occurring in dental plaque of the host.

2. The method of claim 1, wherein the polypeptide is an S. mutans murein hydrolase having a molecular weight of about 65 kDa.

3. The method of claim 2, wherein the polypeptide comprises the amino acid sequence of SEQ. ID NO. 6, or an amino acid sequence having at least 70% identity thereto.

4. The method of claim 2, wherein the polypeptide comprises the amino acid sequence of SEQ. ID NO. 8, or an amino acid sequence having at least 70% identity thereto.

5. The method of claim 1, wherein the host is a human.

6. The method of claim 5, wherein the polypeptide having murein hydrolase activity has murein hydrolase activity effective against one or more members selected from the group consisting of Streptococcus mutans, Streptococcus parasanguis, Streptococcus oralis, Streptococcus sanguis, Streptococcus gordonii, Streptococcus sobrinus, and Actinobacillus actinomycetemcomitans.

7. The method of claim 2, wherein the polypeptide having murein hydrolase activity has murein hydrolase activity against Streptococcus mutans, Streptococcus parasanguis, Streptococcus oralis, Streptococcus sanguis, Streptococcus gordonii, Streptococcus sobrinus, and Actinobacillus actinomycetemcomitans.

8. The method of claim 1, wherein said administering comprises delivering to the oral cavity an oral composition comprising the polypeptide having murein hydrolase activity, and an orally acceptable carrier.

9. The method of claim 8, wherein the oral composition is a mouthwash composition.

10. The method of claim 8, wherein the oral composition is a dentifrice composition.

11. The method of claim 8, wherein the oral composition is a chewing gum.

12. A method for lysing bacteria, comprising contacting the bacteria with a composition comprising a polypeptide having S. mutans murein hydrolase activity.

13. The method of claim 12, wherein the murein hydrolase comprises the amino acid sequence of SEQ. ID NO. 8, or an amino acid sequence having at least 70% identity thereto.

14. The method of claim 13, wherein the murein hydrolase comprises the amino acid sequence of SEQ. ID NO. 8, or an amino acid sequence having at least 80% identity thereto.

15. The method of claim 12, wherein the bacteria include one or more members selected from the group consisting of Streptococcus mutans, Streptococcus parasanguis, Streptococcus oralis, Streptococcus sanguis, Streptococcus gordonii, Streptococcus sobrinus, and Actinobacillus actinomycetemcomitans.

16. A method for controlling dental caries in the oral cavity of a host, comprising administering to the oral cavity of the host an effective amount of a purified protein having a molecular weight of about 65 kDa and having an amino acid sequence having at least about 70% identity with the amino acid sequence of SEQ. I.D. NO. 6.

17. The method of claim 16 wherein the protein is recombinantly-produced.

18. The method of claim 16 which comprises administering an oral composition comprising said protein and an orally-acceptable excipient.

19. The method of claim 17 which comprises administering to the host an oral composition comprising said protein and an orally acceptable excipient.

20. The method of claim 18 wherein the oral composition is a mouthwash composition including water.

21. The method of claim 18 wherein the oral composition is a dentifrice composition including a polishing agent.

22. The method of claim 18 wherein the oral composition is a chewing gum.

23. The method of claim 20 wherein the protein or the polypeptide fragment is recombinantly-produced.

24. A recombinant polypeptide having an amino acid sequence corresponding to the amino acid sequence of SEQ. I.D. NO. 6 or a polypeptide fragment thereof.

25. An oral composition comprising a purified protein having an amino acid sequence having at least 70% identity to SEQ. I.D. NO. 6 and an orally acceptable carrier.

26. The composition of claim 25 which is a mouthwash composition.

27. The composition of claim 25 which is a dentifrice composition.

28. The composition of claim 25 which is a chewing gum.

29. An isolated DNA sequence encoding a protein including an amino acid sequence having at least 70% identity to SEQ. I.D. NO. 6 or a polypeptide fragment thereof.

30. A vector comprising a DNA sequence of claim 29 in operable association with a promoter.

31. A host cell comprising introduced DNA, said introduced DNA including a DNA sequence of claim 29 in operable association with a promoter.

32. A method of producing a polypeptide, comprising culturing a host cell of claim 31 under conditions sufficient to obtain expression of said DNA sequence.

33. The method of claim 32, and also comprising isolating the polypeptide

34. An article of manufacture, comprising:
an oral composition comprising a polypeptide having S. mutans murein hydrolase activity, and an orally acceptable carrier; and
visible indicia relating to use of the oral composition to control dental plaque.

35. The article of claim 34, wherein the polypeptide is a S. mutans murein hydrolase having a molecular weight of about 65 kDa.

36. The article of claim 35, wherein the polypeptide comprises the amino acid sequence of SEQ. ID NO. 8, or an amino acid sequence having at least 70% identity thereto.

37. The article of claim 36, wherein the polypeptide comprises the amino acid sequence of SEQ. ID NO. 8, or an amino acid sequence having at least 80% identity thereto.

38. The article of claim 35, wherein the murein hydrolase has activity against one or more members selected from the group consisting of Streptococcus mutans, Streptococcus parasanguis, Streptococcus oralis, Streptococcus sanguis, Streptococcus gordonii, Streptococcus sobrinus, and Actinobacillus actinomycetemcomitans.

39. The article of claim 37, wherein the polypeptide having murein hydrolase activity has murein hydrolase activity against Streptococcus mutans, Streptococcus parasanguis, Streptococcus oralis, Streptococcus sanguis, Streptococcus gordonii, Streptococcus sobrinus, and Actinobacillus actinomycetemcomitans.

40. The article of claim 32, wherein the oral composition is a mouthwash composition.

41. The article of claim 34, wherein the oral composition is a dentifrice composition.

42. The article of claim 34, wherein the oral composition is a chewing gum.


SEQUENCE LISTING (8 sequences)

SEQ ID NO: 1 - protein, 11 aa, Streptococcus mutans
MSSQAKANNIP

SEQ ID NO: 2 - protein, 11 aa, Streptococcus mutans
  UNSURE (8)..(8)
MQRPTEFXEDK

SEQ ID NO: 3 - protein, 11 aa, Streptococcus mutans
MSRQAKAVNIP

SEQ ID NO: 4 - protein, 11 aa, Streptococcus mutans
MQSPTEFNEDK

SEQ ID NO: 5 - DNA, 2025 bp, Streptococcus mutans
  CDS (96)..(1103)
atagtaaaaatttttcaaaaaatatattacgtaagtattgctaaatatttcttttgtgtttcaatataggtgaaaaaagaaaatgaaggaagattatgaatcaaaaaatagtcgtcatttcgtcattttacatgttaggtgctcattcattttcaaaggcagtatatcataatgataggagtgtgaaacttatgaaaagaattgatattaatcatcaagcacaacgtttttctattcgtaaatatgcatttggagctgcatctgttttaattggctgtgtcttttttctaggtacccaaaatgtttctgcacaagagcagggaactcaattgccagcaagtgaaaacgcagttgtgaacgtggctgaaaattcagttgctatcagccaagcagttgcagataaggcagcaactcaaacaactctaacagaaacaccccaagttgaagttgaggagaaagaaagtaaggtaaatgctcctgctttaaatgtcgatgacaaaggtgcaaaatccaaagaagatgtgaaccctactatttcaaagacagcaagtgaagtggaagcttctgcagtaactgctactgatactaaaaattcaaatccacaagtcaatgttgaaactgactcaagtgaaaaagacgaaaataaaatggtcacctcggctccagctaaggagactgaggcagaacaaaatgagaaagcggtcagagaaaatcttatgcaaagacaagctaaggctgtctcaattccatcgcaaggcaattatgttttccaagaaacaactcctgtaaaaaatgcagccagtatgtccagcccaacccaatttaactttgataaaggagataaggttttttatgataatgttttagaagcggatgggcatcaatggattagctatgtgtcttacagtggtattcgtcgctatgctcctattgctgtgacaattgaagaattgaagcaaaaagaaattgttcagcaaaatttaccggcacaaggaacctatcactttactaaacagcagagcttaaaaatgaagctaaactgtctagtccgacccaattctcgttttacaacggagatcacgttttttatgataaggttttagaagcggatggacatcaatggattagctatgtgtcctacagtggtatccgtcgttatgttgttattggaaagcttacgacacaaccctctccaattgaaactaaagtatcaggtactattgtcatccaaaataaaacggctcaacaattcgatgttgtcatttctaatgcttcaagcaatcaaggcataaaagaggtattagtgccagtttggtcagagcaaaacgggcaggatgacattgtctggtatcaagcaatcaaacaaggtgaaggcgtttataaggtgaccgttaaggtcagtgaccataaaaataatagcggtaactatcatgtccatctttattatcttttggataatggtgaacaaagaggagtcggggcaacaatgactgaggtggaagcaccagagcctgtagaaacaacaggtatcattagcattgccaataagagcagccaaggatttgatgttttgattactaatgcttccagcactcaagacataaaagaggttttagtgccggtttggtcagaacaaaacggacaggacgatattatttggtatcaagcaactaaacaaggcgaaggcgtttataaggtggccgttaaggtcagtgaccataaaaatgacagtggtaactataacattcacctttattatcgccttgtaactggtgaattaaaggttgttggaggaaagacaacgacagtagaagcccctaatagagcaatcttccagcacaaggaacttatgttttcactaataaagttgaggttaaaaatgaggccagaacatctagtccaactcagtttacctttaataaaggagaaagtatttactatgacagtatcttgaatgctgatggacatcaatggattagctatcgttcctacagtggtattcgtcgttatattatcattggttga

SEQ ID NO: 6 - protein, 335 aa, Streptococcus mutans
MNQKIVVISSFYMLGAHSFSKAVYHNDRSVKLMKRIDINHQAQRFSIRKYAFGAASVLIGCVFFLGTQNVSAQEQGTQLPASENAVVNVAENSVAISQAVADKAATQTTLTETPQVEVEEKESKVNAPALNVDDKGAKSKEDVNPTISKTASEVEASAVTATDTKNSNPQVNVETDSSEKDENKMVTSAPAKETEAEQNEKAVRENLMQRQAKAVSIPSQGNYVFQETTPVKNAASMSSPTQFNFDKGDKVFYDNVLEADGHQWISYVSYSGIRRYAPIAVTIEELKQKEIVQQNLPAQGTYHFTKQQSLKMKLNCLVRPNSRFTTEITFFMIRF

SEQ ID NO: 7 - DNA, 1836 bp, Streptococcus mutans
  CDS (1)..(1836)
atgaaaagaattgatattaatcatcaagcacaacgtttttctattcgcaaatatgcatttggagctgcatctgttttaattggctgtgtcttttttctaggtacccaaaatgtttctgcacaagagcagggaactcaattgccagcaagtgaaaacgcagttgtgaacgtggctgaaaattcagttgctatcagccaagcagtttcagataaggcagcagctcaaacaactctaacagaaacaccccaagttgaagttgaggagaaagaaaataaggtaaatgctcctgctttaaatgtcgatgacaaaggtgcaaaatccaaagaagatgtgaaccctactgtttcaaagacagcaagtgaagtggaagcttctgcagtaactgctactgatactaaaaattcaaatccacaagtcaatgttgaaactgactcaaatgaaaaagacgaaaataaaatggtcacctcggctccagctaaggagactgaggcagaacaaaatgagaaagcggtagcagaaaatcttatgcaaagacaagctaaggctgtctcaattccatcgcaaggcaattatgttttccaagaaacaactcctgtaaaaaatgcagccagtatgtccagcccaacccaatttaactttgataaaggagataaggttttttatgataaggttttagaagcggatgggcatcaatggattagctatgtgtcttacagtggtattcgtcgctatgctcctattgctgtgacaattgaagaattgaagcaaaaagaaattgttcagcaaaatttaccggcacaaggaacctatcactttactaaacaagcagacgttaaaaatgaagctaaactgtctagtccgacccaattctcgttttacaacggagatcacgttttttatgataaggttttagaagcggatgggcatcaatggattagctatgtgtcctacagtggtatccgtcgttatgttgttattggaaagcttacgacacaaccctctccaattgaaactaaagtatcaggtactattgccatccaaaataaaacggctcaacaattcgatgttatcatttctaatgtttccagcactcaaggcataaaagaggtattagtgccggtttggtcagagcaaaacgggcaggatgacattgtctggtatcaagcaactaaacaaggcgaaggcgtttataaggtgaccgttaaggtcagtgaccataaaaataacagtggtaactatgacattcacctttattatcgcctttcaactggtgaattaaaggttgttggaggaaagacaactgaggtggaagcaccgaagcctgtagaaacaacaggtatcattagcattgccaataagagcagccaaggatttgatgttttgattactaatgcttccagcactcaaggcataaaagaggtattagtgccagtttggtcagaacaaaacggacaggacgatattatttggtatcaagcaactaaacaaggcgaaggcgtttataaggtgaccgttaaggtcagtgaccataaaaatgacagtggtaactatgacattcacctttattatcgcctttcaactggtgaattaaaggttgttggaggaaagacaacgacagtagaagcccctaatagagtcaatcttccagcacaaggaacttatgttttcactaataaagttgaggttaaaaatgaggccagaacatctagtccaactcagtttacctttaataaaggagaaagtatttactatgacagtatcttgaatgctgatggacatcaatggattagctatcgttcctacagtggtattcgtcgttatattatcattgattga

SEQ ID NO: 8 - protein, 611 aa, Streptococcus mutans
MKRIDINHQAQRFSIRKYAFGAASVLIGCVFFLGTQNVSAQEQGTQLPASENAVVNVAENSVAISQAVSDKAAAQTTLTETPQVEVEEKENKVNAPALNVDDKGAKSKEDVNPTVSKTASEVEASAVTATDTKNSNPQVNVETDSNEKDENKMVTSAPAKETEAEQNEKAVAENLMQRQAKAVSIPSQGNYVFQETTPVKNAASMSSPTQFNFDKGDKVFYDKVLEADGHQWISYVSYSGIRRYAPIAVTIEELKQKEIVQQNLPAQGTYHFTKQADVKNEAKLSSPTQFSFYNGDHVFYDKVLEADGHQWISYVSYSGIRRYVVIGKLTTQPSPIETKVSGTIAIQNKTAQQFDVIISNVSSTQGIKEVLVPVWSEQNGQDDIVWYQATKQGEGVYKVTVKVSDHKNNSGNYDIHLYYRLSTGELKVVGGKTTEVEAPKPVETTGIISIANKSSQGFDVLITNASSTQGIKEVLVPVWSEQNGQDDIIWYQATKQGEGVYKVTVKVSDHKNDSGNYDIHLYYRLSTGELKVVGGKTTTVEAPNRVNLPAQGTYVFTNKVEVKNEARTSSPTQFTFNKGESIYYDSILNADGHQWISYRSYSGIRRYIIID